(19) United States
(12) Patent Application Publication
(10) Pub. No.: US 2025/0352498 A1
(43) Pub. Date: Nov. 20, 2025

(54) PHARMACEUTICAL FORMULATIONS CONTAINING PHENYLBUTYRATE AND METHODS THEREOF

(71) Applicant: AARDVARK THERAPEUTICS INC., San Diego, CA (US)

(72) Inventors: Dvorit SAMID, Los Altos Hills, CA (US); Zhenhuan ZHENG, San Diego, CA (US); Tien-Li LEE, San Diego, CA (US)

(73) Assignee: AARDVARK THERAPEUTICS INC., San Diego, CA (US)

(21) Appl. No.: 19/175,816

(22) Filed: Apr. 10, 2025

Related U.S. Application Data
(60) Provisional application No. 63/633,606, filed on Apr. 12, 2024.

(51) Int. Cl. A61K 31/185; A61K 9/00; A61K 9/107; A61K 47/10; A61K 47/12; A61K 47/34

(52) U.S. Cl. CPC A61K 31/185; A61K 9/0014; A61K 9/107; A61K 47/10; A61K 47/12; A61K 47/34

(57) ABSTRACT

Provided herein are pharmaceutical formulations comprising an optionally 4-substituted phenylbutyrate for improved topical application. In some embodiments, the optionally 4-substituted phenylbutyrate is present at a concentration ranging from about 1-40% by weight. The present disclosure also provides methods of preparing said pharmaceutical formulations, as well as uses and methods of treating subjects affected by inflammatory skin disorder or skin-related disease with said pharmaceutical formulations.

### CROSS-REFERENCE TO RELATED APPLICATIONS

This application claims the benefit of priority of U.S. Provisional Application No. 63/633,606, filed on Apr. 12, 2024, which is incorporated by reference herein in its entirety for all purposes.

### FIELD OF THE INVENTION

The present disclosure provides pharmaceutical formulations comprising an optionally 4-substituted phenylbutyrate for topical application, as well as uses and methods thereof. The present disclosure also provides methods of preparing said pharmaceutical formulations, as well as uses and methods for treating subjects having an inflammatory skin disorder or skin-related disease.

### INTRODUCTION AND SUMMARY

Inflammatory skin conditions and skin diseases are a common ailment affecting millions of adults and children in the United States and globally. Common inflammatory skin conditions include disorders, for example, psoriasis, atopic dermatitis, and eczema. Rare skin disorders having similar features include, for example, epidermolysis bullosa, toxic epidermal necrolysis, and folliculitis. These conditions can all be very uncomfortable for the individual and may result in self-isolation during skin sensitivity and flare-ups. In other instances, skin diseases involving inflammation and/or immune system dysregulation can result in mobility issues, partial disability, and even death if left untreated. For example, psoriatic arthritis, diabetic skin ulcers, and melanoma are skin diseases requiring medical attention.

Psoriasis, which affects an estimated 7.5 million American adults, is a common inflammatory skin condition that causes red, itchy scaly patches, most often on the knees, elbows, trunk, and scalp. Psoriasis is considered an immune system disorder that causes the skin to regenerate faster than normal and involves skin inflammation. There are five types of psoriasis, including plaque psoriasis, guttate psoriasis, pustular psoriasis, inverse psoriasis and erythrodermic psoriasis.

Patients with inflammatory skin conditions have some treatment options, but improvement is desirable and/or a cure for many of these conditions is still lacking. For example, patients with psoriasis are currently treated with corticosteroids, which acts as an anti-inflammatory drug prescribed for autoimmune disease and can be given as an injection into the area, orally, or topically as an ointment, cream, or foam. Other existing treatments for psoriasis include topical therapies, such as vitamin D analogues (e.g., Calcipotriol, to slow skin cell growth), Retinoid, and Coal tar; monoclonal antibody drugs (e.g., Humira); and light therapy, such as sunlight, ultraviolet B broadband and narrowband phototherapy, and excimer lasers. However, no cure exists for psoriasis and existing therapies are lacking.

Phenylbutyrate (PB) has been reported to have activity in a variety of contexts, including metabolic disorder, neurological disease, and blood disorders, when administered systemically. PB is a low-molecular-weight fatty acid that is FDA approved for managing urea cycle disorders in adults and children, when the diseases cannot be managed by changes in diet alone. It has also been used for the treatment of amyotrophic lateral sclerosis (ALS), and it is being explored for use in treating sickle cell disease and thalassemia. PB acts as a nitrogen binding agent via its vivo PB metabolites, a histone deacetylases inhibitor, and a regulator of the hepatocanalicular transporter.

PB is readily available in the form of related 4-substituted derivatives, e.g., wherein the 4-positon is substituted with a halo group. These 4-halo-PB derivatives can have different skin retention and other properties that can be advantageous in certain contexts.

Various formulations of PB have been developed, depending on the indication being addressed, oral, injectable, and topical. However, existing topical formulations of PB have not been found to provide sufficient local concentration, drug release, and/or skin retention, which hinders their utility in treating inflammatory skin disorders. The present disclosure aims to meet the need for improved formulations useful for treating skin conditions such as inflammation, psoriasis, and others, and/or provide other benefits.

Provided herein are pharmaceutical formulations comprising an optionally 4-substituted phenylbutyrate (PB) for use in treating inflammatory skin conditions and other skin-related diseases. In some embodiments, the pharmaceutical formulations described in the instant application provide greater local concentration of the optionally 4-substituted PB and enable a more targeted treatment approach. Accordingly, the following exemplary embodiments are provided.

Embodiment 1 is a pharmaceutical formulation comprising an optionally 4-substituted phenylbutyrate in an emulsion, wherein:
the phenylbutyrate is present at a concentration ranging from about 1-40% by weight,
the emulsion comprises a hydrophobic phase dispersed in an aqueous phase, and the formulation is a topical formulation.

Embodiment 2 is a pharmaceutical formulation comprising an optionally 4-substituted phenylbutyrate in an emulsion, wherein:
the phenylbutyrate is present at a concentration ranging from about 2-40% by weight,
the emulsion comprises a hydrophobic phase and an aqueous phase, and
the formulation is a topical formulation.

Embodiment 3 is a pharmaceutical formulation comprising an optionally 4-substituted phenylbutyrate in an emulsion, wherein:
the emulsion comprises a hydrophobic phase and an aqueous phase,
the aqueous phase comprises water and one or more additional hydrophilic components,
the one or more additional hydrophilic components collectively are present in a ratio of 1:1 or greater by weight relative to the optionally 4-substituted phenylbutyrate,
and the formulation is a topical formulation.

Embodiment 4 is a pharmaceutical formulation comprising an optionally 4-substituted phenylbutyrate in an emulsion, wherein:
the emulsion comprises a hydrophobic phase and an aqueous phase,
the aqueous phase comprises water and one or more additional hydrophilic components,
the formulation has the property of at least about 40% phenylbutyrate release at 6 hours under in vitro release testing conditions,
and the formulation is a topical formulation.

Embodiment 5 is a pharmaceutical formulation comprising an optionally 4-substituted phenylbutyrate in an emulsion, wherein:
the emulsion comprises a hydrophobic phase and an aqueous phase,
the aqueous phase comprises water and one or more additional hydrophilic components,
the formulation has the property of at least about 5% phenylbutyrate skin retention at 6 hours under in vitro skin retention assay conditions,
and the formulation is a topical formulation.

Embodiment 6 is a pharmaceutical formulation comprising an optionally 4-substituted phenylbutyrate in an emulsion, wherein:
the emulsion comprises a hydrophobic phase and an aqueous phase,
the aqueous phase comprises water and one or more additional hydrophilic components,
the formulation has the property of no more than about 12% phenylbutyrate skin permeation at 6 hours under in vitro skin retention assay conditions,
and the formulation is a topical formulation.

Embodiment 7 is a pharmaceutical formulation comprising an optionally 4-substituted phenylbutyrate in an emulsion, wherein:
the emulsion comprises a hydrophobic phase and an aqueous phase,
the aqueous phase comprises water and one or more additional hydrophilic components,
the formulation has the property of at least about 1:1 ratio of phenylbutyrate skin retention to skin permeation at 6 hours under in vitro skin retention assay conditions,
and the formulation is a topical formulation.

Embodiment 8 is the pharmaceutical formulation of embodiment 3 or 4, wherein the optionally 4-substituted phenylbutyrate is present at a concentration ranging from about 1-40% by weight.

Embodiment 9 is the pharmaceutical formulation of any one of embodiments 2-5, wherein the hydrophobic phase is dispersed in the aqueous phase.

Embodiment 10 is the pharmaceutical formulation of embodiment 1 or 2, wherein the aqueous phase comprises water and one or more additional hydrophilic components.

Embodiment 11 is the pharmaceutical formulation of any one of embodiments 4-7, wherein the one or more additional hydrophilic components collectively are present in a ratio of 1:1 or greater by weight relative to the optionally 4-substituted phenylbutyrate.

Embodiment 11.1 is the pharmaceutical formulation of embodiment 11, wherein the one or more additional hydrophilic components is chosen from glycerin, polypropylene glycol, propylene glycol, diethylene glycol monoethyl ether, polyethylene glycol and combinations thereof.

Embodiment 12 is the pharmaceutical formulation of embodiment 1-3 or 5-8, wherein the formulation has the property of at least 40% phenylbutyrate release at 6 hours under in vitro release testing conditions.

Embodiment 13 is the pharmaceutical formulation of any one of the preceding embodiments, wherein the optionally 4-substituted phenylbutyrate is a pharmaceutically acceptable salt.

Embodiment 14 is the pharmaceutical formulation of any one of the preceding embodiments, wherein the optionally 4-substituted phenylbutyrate is a sodium salt.

Embodiment 15 is the pharmaceutical formulation of any one of the preceding embodiments, wherein the optionally 4-substituted phenylbutyrate is a potassium salt.

Embodiment 16 is the pharmaceutical formulation of any one of the preceding embodiments, wherein the optionally 4-substituted phenylbutyrate is a 4-halo phenylbutyrate.

Embodiment 17 is the pharmaceutical formulation of any one of the preceding embodiments, wherein the optionally 4-substituted phenylbutyrate is 4-chlorophenylbutyrate.

Embodiment 18 is the pharmaceutical formulation of any one of embodiments 1-13, wherein the optionally 4-substituted phenylbutyrate is 4-iodophenylbutyrate.

Embodiment 19 is the pharmaceutical formulation of any one of embodiments 1-13, wherein the phenylbutyrate is unsubstituted.

Embodiment 20 is the pharmaceutical formulation of any one of the preceding embodiments, wherein the phenylbutyrate is sodium phenylbutyrate.

Embodiment 21 is the pharmaceutical formulation of any one of the preceding embodiments, wherein the optionally 4-substituted phenylbutyrate is present at a concentration ranging from about 2-40% by weight, 5-40% by weight, or 8-40% by weight.

Embodiment 22 is the pharmaceutical formulation of any one of the preceding embodiments, wherein the optionally 4-substituted phenylbutyrate is present at a concentration ranging from about 1-4% by weight (optionally 2-4% by weight), 4-8% by weight, 8-12% by weight, 12-16% by weight, 16-20% by weight, 20-24% by weight, 24-28% by weight, 28-32% by weight, 32-36% by weight, or 36-40% by weight.

Embodiment 23 is the pharmaceutical formulation of any one of the preceding embodiments, wherein the optionally 4-substituted phenylbutyrate is present at a concentration of about 10% by weight.

Embodiment 24 is the pharmaceutical formulation of any one of the preceding embodiments, wherein the formulation comprises water at a concentration ranging from about 5-80% by weight.

Embodiment 25 is the pharmaceutical formulation of the immediately preceding embodiment, wherein the formulation comprises water at a concentration ranging from about 10-80% by weight, 20-80% by weight, 30-80% by weight, 40-80% by weight, 50-80% by weight, or 60-80% by weight.

Embodiment 26 is the pharmaceutical formulation of embodiment 24, wherein the formulation comprises water at a concentration ranging from about 10-75% by weight, 20-75% by weight, 30-75% by weight, 40-75% by weight, 50-75% by weight, or 60-75% by weight.

Embodiment 27 is the pharmaceutical formulation of embodiment 24, wherein the formulation comprises water at a concentration ranging from about 5-10% by weight, 10-15% by weight, 15-20% by weight, 20-25% by weight, 25-30% by weight, 30-35% by weight, 35-40% by weight, 40-45% by weight, 45-50% by weight, 50-55% by weight, 55-60% by weight, 65-70% by weight, 70-75% by weight, or 75-80% by weight.

Embodiment 28 is the pharmaceutical formulation of any one of the preceding embodiments, wherein the formulation comprises water at a concentration of about 72%, or 72.3+x % by weight, where x is 10, 5, 3, 2, or 1.

Embodiment 29 is the pharmaceutical formulation of any one of the preceding embodiments, wherein the formulation comprises water at a concentration of about 60%, or 60.3+x % by weight, where x is 10, 5, 3, 2, or 1.

Embodiment 30 is the pharmaceutical formulation of any one of the preceding embodiments, wherein the formulation further comprises a nonionic emulsifier.

Embodiment 31 is the pharmaceutical formulation of the immediately preceding embodiment, wherein the nonionic emulsifier comprises glycerol monostearate and/or a PEG stearate.

Embodiment 32 is the pharmaceutical formulation of the immediately preceding embodiment, wherein the PEG stearate is PEG-75 stearate or PEG stearate having a molecular weight of about 3-4 kDa, or about 3.5 kDa.

Embodiment 33 is the pharmaceutical formulation of any one of embodiments 30-32, wherein the nonionic emulsifier comprises isopropyl myristate.

Embodiment 34 is the pharmaceutical formulation of any one of embodiments 30-33, wherein the nonionic emulsifier is present in the formulation at a concentration ranging from about 1-10% by weight, optionally wherein the nonionic emulsifier is a combination of glycerol monostearate and a PEG stearate, further optionally wherein the PEG stearate has a molecular weight of about 3-4 kDa, or about 3.5 kDa.

Embodiment 35 is the pharmaceutical formulation of the immediately preceding embodiment, wherein the nonionic emulsifier is present in the formulation at a concentration ranging from about 1-2% by weight, 2-4% by weight, 4-6% by weight, 6-8% by weight, or 8-10% by weight, optionally wherein the nonionic emulsifier is a combination of glycerol monostearate and a PEG stearate, further optionally wherein the PEG stearate has a molecular weight of about 3-4 kDa, or about 3.5 kDa.

Embodiment 36 is the pharmaceutical formulation of the immediately preceding embodiment, wherein the nonionic emulsifier is present in the formulation at a concentration of about 5% by weight, optionally wherein the nonionic emulsifier is a combination of glycerol monostearate and a PEG stearate, further optionally wherein the PEG stearate has a molecular weight of about 3-4 kDa, or about 3.5 kDa.

Embodiment 37 is the pharmaceutical formulation of any one of the preceding embodiments, wherein the formulation further comprises an emollient.

Embodiment 38 is the pharmaceutical formulation of the immediately preceding embodiment, wherein the emollient comprises stearyl alcohol.

Embodiment 39 is the pharmaceutical formulation of embodiment 37, wherein the emollient comprises cetyl alcohol or a combination of cetyl and stearyl alcohols.

Embodiment 40 is the pharmaceutical formulation of any one of embodiments 37-39, wherein the emollient is present in the formulation at a concentration ranging from about 1-10% by weight, optionally wherein the emollient is stearyl alcohol.

Embodiment 41 is the pharmaceutical formulation of the immediately preceding embodiment, wherein the nonionic emulsifier is present in the formulation at a concentration ranging from about 1-2% by weight, 2-4% by weight, 4-6% by weight, 5-7% by weight, 6-8% by weight, or 8-10% by weight, optionally wherein the emollient is stearyl alcohol.

Embodiment 42 is the pharmaceutical formulation of the immediately preceding embodiment, wherein the nonionic emulsifier is present in the formulation at a concentration of about 3% by weight, optionally wherein the emollient is stearyl alcohol.

Embodiment 43 is the pharmaceutical formulation of embodiment 41, wherein the nonionic emulsifier is present in the formulation at a concentration of about 6% by weight, optionally wherein the emollient is stearyl alcohol.

Embodiment 44 is the pharmaceutical formulation of any one of the preceding embodiments, wherein the formulation further comprises an antioxidant.

Embodiment 45 is the pharmaceutical formulation of the immediately preceding embodiment, wherein the antioxidant comprises a thiosulfate.

Embodiment 46 is the pharmaceutical formulation of the immediately preceding embodiment, wherein the thiosulfate is sodium thiosulfate.

Embodiment 47 is the pharmaceutical formulation of any one of embodiments 44-46, wherein the antioxidant is present in the formulation at a concentration ranging from about 0.1-1% by weight, optionally wherein the antioxidant is sodium thiosulfate.

Embodiment 48 is the pharmaceutical formulation of the immediately preceding embodiment, wherein the antioxidant is present in the formulation at a concentration ranging from about 0.1-0.2% by weight, 0.2-0.4% by weight, 0.4-0.6% by weight, 0.6-0.8% by weight, or 0.8-1% by weight, optionally wherein the antioxidant is sodium thiosulfate.

Embodiment 49 is the pharmaceutical formulation of the immediately preceding embodiment, wherein the antioxidant is present in the formulation at a concentration ranging from about 0.05-0.2% by weight or about 0.1% by weight, optionally wherein the antioxidant is sodium thiosulfate.

Embodiment 50 is the pharmaceutical formulation of any one of the preceding embodiments, wherein the formulation further comprises a preservative.

Embodiment 51 is the pharmaceutical formulation of the immediately preceding embodiment, wherein the preservative comprises a benzoate.

Embodiment 52 is the pharmaceutical formulation of the immediately preceding embodiment, wherein the thiosulfate is sodium benzoate.

Embodiment 53 is the pharmaceutical formulation of any one of embodiments 50-52, wherein the preservative is present in the formulation at a concentration ranging from about 0.1-1% by weight, optionally wherein the preservative is sodium benzoate.

Embodiment 54 is the pharmaceutical formulation of the immediately preceding embodiment, wherein the preservative is present in the formulation at a concentration ranging from about 0.05-0.15% by weight, 0.15-0.25% by weight, 0.25-0.35% by weight, or 0.35-0.5% by weight, optionally wherein the preservative is sodium benzoate.

Embodiment 55 is the pharmaceutical formulation of the immediately preceding embodiment, wherein the preservative is present in the formulation at a concentration ranging from about 0.15-0.25% by weight or about 0.2% by weight, optionally wherein the preservative is sodium benzoate.

Embodiment 56 is the pharmaceutical formulation of any one of the preceding embodiments, wherein the formulation further comprises at least one additional excipient.

Embodiment 57 is the pharmaceutical formulation of the immediately preceding embodiment, wherein the at least one additional excipient comprises a PEG, optionally wherein the PEG is PEG400; a glycerol polyethylene glycol hydroxy stearate; a hydrogenated ethoxylated castor oil, optionally wherein the hydrogenated ethoxylated castor oil is Peg-60 hydrogenated castor oil; and/or glycerin.

Embodiment 58 is the pharmaceutical formulation of any one of embodiments 56-57, wherein the at least one additional excipient is present in the formulation at a concentration ranging from about 0.1-0.2% by weight, 0.2-0.4% by weight, 0.4-0.6% by weight, 0.6-0.8% by weight, 0.8-1% by weight, 1-2% by weight, 2-4% by weight, 4-6% by weight, 5-7% by weight, 6-8% by weight, or 8-10% by weight.

Embodiment 59 is the pharmaceutical formulation of any one of the preceding embodiments, wherein the topical formulation is a cream, ointment, gel, spray, or foam, or wherein the topical formulation is contained in a patch or dressing, optionally wherein the patch is an adhesive patch or the dressing is a non-adhesive dressing, an occlusive dressing, or a non-occlusive dressing.

Embodiment 60 is the pharmaceutical formulation of any one of the preceding embodiments, wherein the topical formulation is a cream.

Embodiment 61 is the pharmaceutical formulation of any one of the preceding embodiments, wherein the formulation comprises less than about 0.01%, 0.001%, 0.0005%, or 0.0003% by weight of

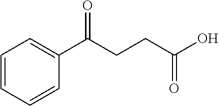 [SMILES: O=C(O)CCC(=O)c1ccccc1]

(related compound A) and salts thereof.

Embodiment 62 is the pharmaceutical formulation of any one of the preceding embodiments, wherein the formulation undergoes less than about 0.01%, 0.001%, 0.0005%, or 0.0003% conversion by weight of the phenylbutyrate to

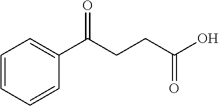 [SMILES: O=C(O)CCC(=O)c1ccccc1]

(related compound A) and salts thereof upon storage at 60° C. for 21 days.

Embodiment 63 is the pharmaceutical formulation of any one of the preceding embodiments, wherein the formulation has the property of at least about 50%, 60%, or 70% phenylbutyrate release at 6 hours under in vitro release testing conditions.

Embodiment 64 is the pharmaceutical formulation of any one of the preceding embodiments, wherein the formulation has the property of at least about 40%, 50%, 60%, or 70% phenylbutyrate release at 5 hours under in vitro release testing conditions.

Embodiment 65 is the pharmaceutical formulation of any one of the preceding embodiments, wherein the formulation has the property of at least about 40%, 50%, 60%, or 70% phenylbutyrate release at 4 hours under in vitro release testing conditions.

Embodiment 66 is the pharmaceutical formulation of any one of the preceding embodiments, wherein the formulation has the property of at least about 40%, 50%, 60%, or 70% phenylbutyrate release at 3 hours under in vitro release testing conditions.

Embodiment 67 is the pharmaceutical formulation of any one of the preceding embodiments, wherein the formulation has the property of at least about 40%, 50%, or 60% phenylbutyrate release at 2 hours under in vitro release testing conditions.

Embodiment 68 is the pharmaceutical formulation of any one of the preceding embodiments, wherein the formulation has the property of at least about 70% phenylbutyrate release at 2 hours under in vitro release testing conditions.

Embodiment 69 is the pharmaceutical formulation of any one of the preceding embodiments, wherein the formulation has the property of at least about 10%, 15%, or 20% phenylbutyrate skin retention at 6 hours under in vitro skin retention assay conditions.

Embodiment 70 is the pharmaceutical formulation of any one of the preceding embodiments, wherein the formulation has the property of at least about 15%, 16%, 17%, 18%, 19%, or 20% phenylbutyrate skin retention at 6 hours under in vitro skin retention assay conditions.

Embodiment 71 is the pharmaceutical formulation of any one of the preceding embodiments, wherein the formulation has the property of at least about 15% phenylbutyrate skin retention at 6 hours under in vitro skin retention assay conditions.

Embodiment 72 is the pharmaceutical formulation of any one of the preceding embodiments, wherein the formulation has the property of no more than about 10%, 8%, 6%, or 4% phenylbutyrate skin permeation at 6 hours under in vitro skin retention assay conditions.

Embodiment 73 is the pharmaceutical formulation of any one of the preceding embodiments, wherein the formulation has the property of no more than about 10%, 9%, 8%, 7%, 6%, 5%, or 4% phenylbutyrate skin permeation at 6 hours under in vitro skin retention assay conditions.

Embodiment 74 is the pharmaceutical formulation of any one of the preceding embodiments, wherein the formulation has the property of no more than about 4% phenylbutyrate skin permeation at 6 hours under in vitro skin retention assay conditions.

Embodiment 75 is the pharmaceutical formulation of any one of the preceding embodiments, wherein the formulation has the property of at least about 2:1, 3:1, or 4:1 ratio of phenylbutyrate skin retention to skin permeation at 6 hours under in vitro skin retention assay conditions.

Embodiment 76 is the pharmaceutical formulation of any one of the preceding embodiments, wherein the formulation has the property of at least about 4:1 ratio of phenylbutyrate skin retention to skin permeation at 6 hours under in vitro skin retention assay conditions.

Embodiment 77 is the pharmaceutical formulation of any one of the preceding embodiments, wherein the formulation has a pH in the range of about 4-8, 5-7, or 6.2-7.2.

Embodiment 78 is the pharmaceutical formulation of any one of the preceding embodiments, wherein the formulation has a pH in the range of about 6.5-7.

Embodiment 79 is the pharmaceutical formulation of any one of the preceding embodiments, wherein the formulation has a viscosity in the range of about 3500-6500 cps.

Embodiment 80 is the pharmaceutical formulation of any one of the preceding embodiments, wherein the formulation has a viscosity in the range of about 4500-5500 cps.

Embodiment 81 is the pharmaceutical formulation of any one of the preceding embodiments, wherein the formulation is stable under stress storage conditions comprising a temperature of at least 25±2° C. and a relative humidity (RH) at least 60±5% for at least 3 months.

Embodiment 82 is the pharmaceutical formulation of any one of the preceding embodiments, wherein the formulation is stable under stress storage conditions comprising a temperature of at least 40±2° C. and a relative humidity (RH) at least 75±5% for at least 3 months.

Embodiment 83 is the pharmaceutical formulation of any one of the preceding embodiments, wherein the formulation is stable under stress storage conditions comprising a temperature of at least 60° C. for at least 7 days.

Embodiment 84 is the pharmaceutical formulation of any one of the preceding embodiments, wherein the formulation is stable under stress storage conditions comprising a temperature of at least 60° C. for at least 21 days.

Embodiment 85 is a method of treating skin inflammation, comprising applying the pharmaceutical formulation of any one of embodiments 1-84 topically to a subject in need thereof.

Embodiment 86 is a method of treating psoriasis, comprising applying the pharmaceutical formulation of any one of embodiments 1-84 topically to a subject having psoriasis.

Embodiment 87 is a method of treating a skin condition, comprising applying the pharmaceutical formulation of any one of embodiments 1-84 topically to a subject having the skin condition.

Embodiment 88 is a method of delivering an optionally 4-substituted phenylbutyrate to the skin of a subject, comprising applying the pharmaceutical formulation of any one of embodiments 1-84 topically to the subject.

Embodiment 89 is use of the pharmaceutical formulation of any one of embodiments 1-84 for the manufacture of a medicament.

Embodiment 90 is use of the pharmaceutical formulation of any one of embodiments 1-84 for the manufacture of a medicament for treating skin inflammation.

Embodiment 91 is use of the pharmaceutical formulation of any one of embodiments 1-84 for the manufacture of a medicament for treating psoriasis.

Embodiment 92 is use of the pharmaceutical formulation of any one of embodiments 1-84 for the manufacture of a medicament for treating a skin condition.

Embodiment 93 is the pharmaceutical formulation of any one of embodiments 1-84 for use in therapy.

Embodiment 94 is the pharmaceutical formulation of any one of embodiments 1-84 for use in treating skin inflammation.

Embodiment 95 is the pharmaceutical formulation of any one of embodiments 1-84 for use in treating psoriasis.

Embodiment 96 is the pharmaceutical formulation of any one of embodiments 1-84 for use in treating a skin condition.

Embodiment 97 is the method, use, or pharmaceutical formulation for use of any one of embodiments 85, 87, 88-90, 92-94, or 96, for treating an inflammatory skin disorder, epidermolysis bullosa, epidermolysis bullosa simplex, atopic dermatitis, eczema, seborrheic dermatitis, rosacea, toxic epidermal necrolysis, Stevens Johnson syndrome, Lyell syndrome, erythema multiforme, Necrobiosis lipoidica, Peeling skin syndrome, Ichthyosis, Neurodermatitis, Pemphigus, folliculitis, skin inflammation caused by a cancer therapy optionally wherein the cancer therapy comprises an epidermal growth factor inhibitor, skin cancer, melanoma, squamous cell carcinoma, basal cell carcinoma, Merkel cell carcinoma, dermatofibrosarcoma protuberans, actinic keratosis, sebaceous carcinoma, a skin wound, a skin ulcer, a venous skin ulcer, a diabetic skin ulcer, arthritis, osteoarthritis, psoriatic arthritis, itching, pruritus, cholestatic pruritus, skin aging, skin wrinkles, a gram-positive bacterial skin infection, a staphylococcal skin infection, an S. aureus skin infection, acne, and/or advanced glycation end-product accumulation.

Embodiment 98 is a method of preparing the pharmaceutical formulation of any one of embodiments 1-84, comprising emulsifying and homogenizing a hydrophobic phase and an aqueous phase to produce said formulation.

### BRIEF DESCRIPTION OF THE DRAWINGS

FIGS. 1A-1B are tables listing the ingredients of formulation 1, 2, or 3. Active pharmaceutical ingredients, API; cetostearyl alcohol, CSA; and isopropyl myristate, IPM.

FIG. 2A illustrates an exemplary workflow of methods of preparing formulations described herein. FIG. 2B is a schematic of the one-step mixing method for preparing formulations described herein. FIG. 2C is a timeline schematic describing the timing of steps in the method of preparing formulations described herein.

FIG. 3A-3C are exemplary ultra-performance liquid chromatographic (UPLC) chromatogram results for formulation impurities.

FIG. 4 is a schematic of the in vitro skin retention assay apparatus as described in Example 3.

FIG. 5A is a schematic of the in vitro release testing apparatus as described in Example 4.

FIGS. 5B-5C illustrate exemplary results for in vitro release testing of formulations 1, 2, and 3. FIG. 5B shows the correlations between released phenylbutyrate and the square root of sample time. FIG. 5C shows the percent phenylbutyrate release over time.

FIGS. 6A-6D are tables depicting stress storage stability results for formulation 1, 2, and/or 3, where sodium phenylbutyrate, impurities, and/or mass balance are assessed. FIG. 6A depicts stress storage stability results for formulation 1 under either 25±2° C. and a relative humidity (RH) at least 60±5% or 40±2° C. and a RH at least 75=5% for 1 month. FIG. 6B depicts stress storage stability results for formulation 1, 2, and 3 under 60° C. for 7 days. FIG. 6C depicts stress storage stability results for formulation 1, 2, and 3 under 60° C. for 21 days. FIG. 6D depicts stress storage stability results for formulation 1 under either 25±2° C. and a relative humidity (RH) at least 60±5% for 1, 3, 6, or 12 months or 40±2° C. and a RH at least 75±5% for 1, 3, or 6 months.

FIG. 7A is a table listing the ingredients of formulation 1.1. Cetostearyl alcohol, CSA. FIG. 7B shows an exemplary timeline schematic of the method for preparing formulation 1.1, as described generally in Example 2. FIG. 7C is a plot of phenylbutyrate content at the described stages of the formulation 1.1 manufacturing process. FIG. 7D is a table listing the ingredients of formulation 1.2. FIG. 7E shows an exemplary timeline schematic of the method for preparing formulation 1.2, as described generally in Example 2. FIG. 7F is a plot of phenylbutyrate content at the described stages of the formulation 1.2 manufacturing process. FIGS. 7G-7H illustrate exemplary results for in vitro release testing of formulations 1.2. FIG. 7G shows the correlations between released phenylbutyrate and the square root of sample time. FIG. 7H shows the percent phenylbutyrate release over time.

FIG. 8 is a table listing the solubility of sodium phenylbutyrate in various solvents and excipients. See FIGS. 9A-9B for structure/generic terminology of the solvents and excipients. Tween20 is also known as polysorbate 20.

FIG. 9A is a table listing various emollient and emulsifier excipients and their structure.

FIG. 9B is a table listing various surfactant and penetration enhancer/rheology modifier excipients, a solvent, and additional hydrophilic components and their structure.

FIG. 10 is a table listing the ingredients of formulations 2.1, 2.2, 2.3, 2.4, and 2.5. API, Active pharmaceutical ingredients. See FIGS. 9A-9B for structure/generic terminology of various components.

### DETAILED DESCRIPTION OF CERTAIN EMBODIMENTS

Reference will now be made in detail to certain embodiments of the invention. While the invention will be described in conjunction with such embodiments, it will be understood that they are not intended to limit the invention to those embodiments. On the contrary, the invention is intended to cover all alternatives, modifications, and equivalents, which may be included within the invention as defined by the appended claims.

Before describing the present teachings in detail, it is to be understood that the disclosure is not limited to specific compositions or process steps, as such may vary. It should be noted that, as used in this specification and the appended claims, the singular form “a,” “an,” and “the” include plural references unless the context clearly dictates otherwise. Thus, for example, reference to “an emulsifier” includes a plurality of emulsifiers, and the like. “Or” is used in the inclusive sense, equivalent to “and/or,” unless the context clearly dictates otherwise.

Numeric ranges are inclusive of the numbers defining the range. Measured and measurable values are understood to be approximate, taking into account significant digits and the error associated with the measurement. Also, the use of “comprise,” “comprises,” “comprising,” “contain,” “contains,” “containing,” “include,” “includes,” and “including” are not intended to be limiting. It is to be understood that both the foregoing general description and detailed description are exemplary and explanatory only and are not restrictive of the teachings.

Unless specifically noted in the specification, embodiments in the specification that recite “comprising” various components are also contemplated as “consisting of” or “consisting essentially of” the recited components; embodiments in the specification that recite “consisting of” various components are also contemplated as “comprising” or “consisting essentially of” the recited components; and embodiments in the specification that recite “consisting essentially of” various components are also contemplated as “consisting of” or “comprising” the recited components (this interchangeability does not apply to the use of these terms in the claims). Any “pharmaceutical formulation” described herein is also contemplated as a “composition” or “formulation.”

The section headings used herein are for organizational purposes and are not to be construed as limiting the disclosed subject matter in any way. In the event that any document or other material incorporated by reference contradicts any explicit content of this specification, including definitions, this specification controls.

### I. Definitions

As used herein, “phenylbutyrate,” “PB,” “phenylbutyric acid,” and “PBA,” are used interchangeably. Phenylbutyrate is understood to include 4-substituted forms of phenylbutyrate as well as unsubstituted phenylbutyrate, and both free acid and salt forms.

As used herein, “skin retention” refers to the amount, percentage, ratio, or otherwise collective total of the active pharmaceutical ingredient or drug (e.g., phenylbutyrate) present in the skin after application of a topical formulation comprising the active pharmaceutical ingredient or drug (e.g., phenylbutyrate). Skin retention can be evaluated using in vitro skin retention assay conditions. Unless otherwise indicated, for skin retention expressed using a percentage, the percentage is by weight.

As used herein, “skin permeation” refers to the amount, percentage, ratio, or otherwise collective total of the active pharmaceutical ingredient or drug that has permeated the skin (e.g., phenylbutyrate) and is available systemically, as represented by the collected samples that permeated the skin during the in vitro skin retention assay. Unless otherwise indicated, for skin permeation expressed using a percentage, the percentage is by weight.

The term “in vitro skin retention assay conditions” refers to assay conditions comprising preparing a full thickness cadaver skin cut into a round shape having a radius of 1.6 cm and mounting the cut cadaver skin onto a modified Franz cell system equipped with a water bath set to 37° C., where the cadaver skin is clamped between the donor chamber and the receptor chamber, phosphate buffer is used to fill the sampling port, and about 0.5 g of topical formulation comprising phenylbutyrate (PB) is applied to the skin cadaver, along with a release liner to ensure even formulation application. Hourly permeation samples (about 1 mL) are collected from the sampling port (up to 6 hours) and are prepared for subsequent HPLC analysis of phenylbutyrate. After wiping the formulation from the skin with a kimwipe, the used kimwipe, treated cadaver skin sample, and collected permeation samples are mixed with 100% methanol solution to extract active pharmaceutical ingredient or drug (e.g., phenylbutyrate) for quantification of “on skin,” skin retention, and skin permeation amounts. Example 3 provides an example in which in vitro skin retention assay conditions are used.

As used herein, “in vitro release testing conditions” refers to assay conditions comprising applying about 0.5 g of topical formulation comprising phenylbutyrate (PB) to a nylon membrane (0.45 μm) in a Franz Vertical Diffusion Cell apparatus, which is warmed to 32±1° C., and collecting hourly samples of about 1 mL for subsequent HPLC analysis of phenylbutyrate. Example 4 provides an example in which in vitro release testing conditions are used.

As used herein, the term “additional hydrophilic component” refers to a component of the aqueous phase other than water and the optionally 4-substituted phenylbutyrate.

### II. Pharmaceutical Formulations

### A. Phenylbutyrate

Provided herein are pharmaceutical formulations comprising an optionally 4-substituted phenylbutyrate. In some embodiments, the pharmaceutical formulations comprising an optionally 4-substituted phenylbutyrate, wherein the phenylbutyrate is present at a concentration ranging from about 1-40% by weight. In some embodiments, the pharmaceutical formulations comprising an optionally 4-substituted phenylbutyrate, wherein the phenylbutyrate is present at a concentration ranging from about 2-40% by weight.

Phenylbutyrate (also known as 4-phenylbutyrate; 4-phenylbutyric acid; 4-PBA) is an aromatic fatty acid. Structurally, PB comprises a phenyl ring and a butyric acid moiety and commonly exists as a PB salt, such as sodium phenylbutyrate. Pharmaceutically acceptable phenylbutyrate salts include, for example, sodium and potassium salts. As used herein “optionally 4-substituted phenylbutyrate” refers to a 4-(4-substituted-phenyl)-butyrate wherein the optional substituent is on the phenyl ring.

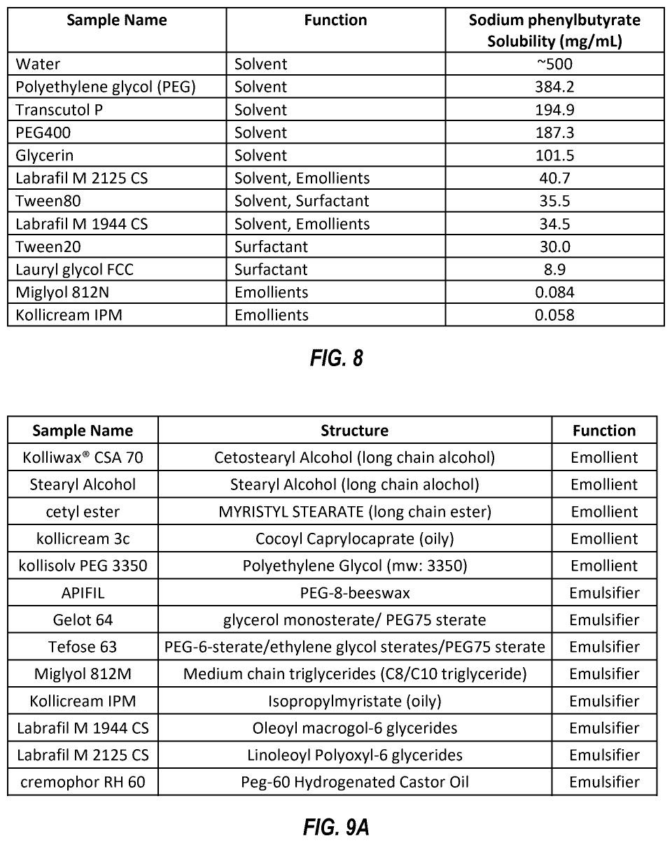

Phenylbutyrate has a wide range of solubilities in different solvents and excipients, as shown in FIG. 8. In some embodiments, the solubility of phenylbutyrate greater than 100 mg/mL in hydrophilic solvent. In some embodiments, the solubility of phenylbutyrate less than 100 mg/mL in hydrophobic excipients. In some embodiments, the solubility of phenylbutyrate in a solvent is between about 30 mg/mL and about 500 mg/mL. In some embodiments, the solubility of phenylbutyrate in a solvent is between 30-500 mg/mL, 50-400 mg/mL, 70-300 mg/mL, or 100-200 mg/mL. In some embodiments, the solubility of phenylbutyrate in a solvent is about 30 mg/mL. In some embodiments, the solubility of phenylbutyrate in a solvent is about 500 mg/mL. In some embodiments, the solubility of phenylbutyrate in a solvent is greater than 33 mg/mL. Sodium phenyl butyrate is highly soluble in hydrophilic solvents and excipients and poorly soluble in hydrophobic solvents and excipients.

Phenylbutyrate (PB) inhibits the development of endoplasmic reticulum stress, the action of histone deacetylases and as a regulator of the hepatocanalicular transporter. This has stimulated interest in sodium phenylbutyrate as an anti-cancer agent. PB is also a chemical chaperone, which has spurred interest in understanding its use in protein misfolding diseases, such as cystic fibrosis.

Without being bound to theory, phenylbutyrate (PB) may have novel mechanisms of action not previously recognized and/or fully appreciated. PB may act as a chemical chaperone to reduce endoplasmic reticulum (ER) stress and the unfolded protein response (UPR), which are implicated in a broad spectrum of skin disorders, including psoriasis. PB may also target other pathways associated with inflammatory skin disorders, leading to keratinocyte differentiation and inhibition of inflammatory cytokines. PB may also have anti-microbial activity against bacteria, fungi, or viruses associated with skin disorders and diseases.

In some embodiments, provided herein are pharmaceutical formulations comprising an optionally 4-substituted phenylbutyrate, wherein the phenylbutyrate is present at a concentration ranging from about 1% to about 40% by weight. In some embodiments, provided herein are pharmaceutical formulations comprising an optionally 4-substituted phenylbutyrate, wherein the phenylbutyrate is present at a concentration ranging from about 2% to about 40% by weight.

In some embodiments, provided herein are pharmaceutical formulations comprising an optionally 4-substituted phenylbutyrate, wherein the phenylbutyrate is present at a concentration ranging from about 1% to about 40%, 2% to about 38%, 3% to about 36%, 4% to about 34%, 5% to about 32%, 6% to about 30%, 7% to about 28%, 8% to about 26%, 9% to about 24%, 10% to about 22%, 11% to about 18%, or 12% to about 16% by weight. In some embodiments, the optionally 4-substituted phenylbutyrate is present at a concentration of at least about 1%, about 2%, 3%, 4%, 5%, 6%, 7%, 8%, 9%, or 10% by weight. In some embodiments, the pharmaceutical formulation comprises an optionally 4-substituted phenylbutyrate, wherein the phenylbutyrate is present at a concentration of about 5% by weight. In some embodiments, the pharmaceutical formulation comprises an optionally 4-substituted phenylbutyrate, wherein the phenylbutyrate is present at a concentration of about 10% by weight.

In some embodiments, the optionally 4-substituted phenylbutyrate is present at a concentration ranging from about 2-40% by weight, 5-40% by weight, or 8-40% by weight. In some embodiments, the optionally 4-substituted phenylbutyrate is present at a concentration ranging from about 1-4% by weight (optionally 2-4% by weight), 4-8% by weight, 8-12% by weight, 12-16% by weight, 16-20% by weight, 20-24% by weight, 24-28% by weight, 28-32% by weight, 32-36% by weight, or 36-40% by weight. In some embodiments, the optionally 4-substituted phenylbutyrate is present at a concentration of about 10% by weight.

In some embodiments, the optionally 4-substituted phenylbutyrate is a pharmaceutically acceptable salt. In some embodiments, the optionally 4-substituted phenylbutyrate is a sodium salt. In some embodiments, the optionally 4-substituted phenylbutyrate is a potassium salt. In some embodiments, the optionally 4-substituted phenylbutyrate is a 4-halo phenylbutyrate. In some embodiments, the optionally 4-substituted phenylbutyrate is 4-chlorophenylbutyrate. In some embodiments, the optionally 4-substituted phenylbutyrate is 4-iodophenylbutyrate. In some embodiments, the phenylbutyrate is unsubstituted. In some embodiments, the phenylbutyrate is sodium phenylbutyrate.

### B. Additional Components

Provided herein are pharmaceutical formulations comprising an optionally 4-substituted phenylbutyrate in an emulsion, wherein the phenylbutyrate is present at a concentration ranging from about 1% to about 40% by weight, and wherein the emulsion comprises a hydrophobic phase and an aqueous phase. In some embodiments, the aqueous phase comprises water and one or more additional hydrophilic components. In some embodiments, the one or more additional hydrophilic components are present, e.g., collectively, in a ratio of 1:1 or greater by weight relative to the optionally 4-substituted phenylbutyrate. The one or more additional hydrophilic components may comprise any one or more of the components described elsewhere herein that are water-soluble. In some embodiments, the one or more additional hydrophilic components comprise any one or more of an alcohol, a glycol, a polyalkylene glycol (e.g., polyethylene glycol or polypropylene glycol), glycerin, sodium thiosulfate, or sodium benzoate. In some embodiments, the one or more additional hydrophilic components are pharmaceutically acceptable.

In some embodiments, provided herein is a pharmaceutical formulation comprising an optionally 4-substituted phenylbutyrate in an emulsion, wherein the emulsion comprises a hydrophobic phase and an aqueous phase, and wherein the aqueous phase comprises water. In some embodiments, the formulation comprises water at a concentration ranging from about 5% to about 80% by weight. In some embodiments, the formulation comprises water at a concentration ranging from about 5% to about 80%, 15% to about 79%, 25% to about 78%, 35% to about 77%, 45% to about 76%, 55% to about 75%, 65% to about 74%, or 70% to about 73% by weight. In some embodiments, the formulation comprises water at a concentration of about 72%, or 72.3+x % by weight, where x is 10, 5, 3, 2, or 1. In some embodiments, the formulation comprises water at a concentration of about 60%, or 60.3+x % by weight, where x is 10, 5, 3, 2, or 1.

In some embodiments, provided herein is a pharmaceutical formulation comprising an optionally 4-substituted phenylbutyrate in an emulsion, wherein the emulsion comprises a hydrophobic phase and an aqueous phase, and wherein the aqueous phase comprises water. In some embodiments, the formulation comprises water at a concentration ranging from about 5-80% by weight. In some embodiments, the formulation comprises water at a concentration ranging from about 10-80% by weight, 20-80% by weight, 30-80% by weight, 40-80% by weight, 50-80% by weight, or 60-80% by weight. In some embodiments, the formulation comprises water at a concentration ranging from about 10-75% by weight, 20-75% by weight, 30-75% by weight, 40-75% by weight, 50-75% by weight, or 60-75% by weight. In some embodiments, the formulation comprises water at a concentration ranging from about 5-10% by weight, 10-15% by weight, 15-20% by weight, 20-25% by weight, 25-30% by weight, 30-35% by weight, 35-40% by weight, 40-45% by weight, 45-50% by weight, 50-55% by weight, 55-60% by weight, 65-70% by weight, 70-75% by weight, or 75-80% by weight. In some embodiments, the formulation comprises water at a concentration of about 72%, or 72.3+x % by weight, where x is 10, 5, 3, 2, or 1. In some embodiments, the formulation comprises water at a concentration of about 60%, or 60.3+x % by weight, where x is 10, 5, 3, 2, or 1.

In some embodiments, provided herein is a pharmaceutical formulation comprising an optionally 4-substituted phenylbutyrate in an emulsion, wherein the emulsion comprises a hydrophobic phase and an aqueous phase, and wherein the aqueous phase comprises water and one or more additional hydrophilic components. In some embodiments, the one or more additional hydrophilic components are present, e.g., collectively, in a ratio of 1:1 or greater by weight relative to the optionally 4-substituted phenylbutyrate. For example, in a formulation comprising 10% phenylbutyrate, two or more additional hydrophilic components may be present in concentrations that sum to 10% or more. Also contemplated are embodiments in which an individual additional hydrophilic component is present in a ratio of 1:2 or greater by weight relative to the optionally 4-substituted phenylbutyrate (e.g., at least 5% if the phenylbutyrate is present at 10%).

In some embodiments, the pharmaceutical formulation comprises sodium phenylbutyrate, Gelot™ 64 (Mono and Diglycerides and Polyoxyl stearate), Kolliwax® CSA 70 (cetostearyl alcohol), glycerin, 20% sodium thiosulfate solution, sodium benzoate, and water. In some embodiments, the formulation consists of sodium phenylbutyrate, Gelot™ 64, Kolliwax® CSA 70, glycerin, 20% sodium thiosulfate solution, sodium benzoate, and water.

In some embodiments, sodium phenylbutyrate, glycerin, sodium thiosulfate solution 20% and sodium benzoate are water soluble. In some embodiments, Gelot™ 64 and Kolliwax® CSA 70 are emulsifiers. In some embodiments, Gelot™ 64 has a melting point between 55.5° C. and 62.5° C. and Kolliwax® CSA 70 has a melting point between 49° C. and 56° C.

In some embodiments, the pharmaceutical formulation comprises sodium phenylbutyrate, Gelot™ 64 (Mono and Diglycerides and Polyoxyl stearate), stearyl alcohol, glycerin, 20% sodium thiosulfate solution, sodium benzoate, and water. In some embodiments, the formulation consists of sodium phenylbutyrate, Gelot™ 64, Kolliwax® CSA 70, glycerin, 20% sodium thiosulfate solution, sodium benzoate, and water.

In some embodiments, the pharmaceutical formulation comprises sodium phenylbutyrate, Gelot™ 64, Kolliwax® CSA 70, PEG 400, isopropyl myristate (IPM), RH60, glycerin, 20% sodium thiosulfate solution, sodium benzoate, and water. In some embodiments, the formulation consists of sodium phenylbutyrate, Gelot™ 64, Kolliwax® CSA 70, PEG 400, IPM, RH60, glycerin, 20% sodium thiosulfate solution, sodium benzoate, and water.

In some embodiments, the pharmaceutical formulation comprises sodium phenylbutyrate, Gelot™ 64, Kolliwax® CSA 70, Carbomer 974P, RH40, glycerin, sodium thiosulfate (e.g., as 20% sodium thiosulfate solution), sodium benzoate, and water. In some embodiments, the formulation consists of sodium phenylbutyrate, Gelot™ 64, Kolliwax® CSA 70, Carbomer 974P, RH40, glycerin, sodium thiosulfate (e.g., as 20% sodium thiosulfate solution), sodium benzoate, and water.

### 1. Emulsifiers

Emulsifiers help form and stabilize oil-in water emulsions (e.g., pharmaceutical and topical formulations) by reducing the difference in surface tension between liquids that do not typically mix together. Emulsifiers also maintain and improve certain characteristics that are useful for formulation function and consumer appeal.

In some embodiments, the formulation further comprises a nonionic emulsifier. Examples of nonionic emulsifiers include, but are not limited to PEG-8-beeswax (e.g., APIFIL), glycerol monostearate/PEG75 stearate (e.g., Gelot 64), PEG-6-sterate/ethylene glycol stearates/PEG75 stearate (e.g., Tefose 63), medium chain triglycerides (C8/C10 triglyceride) (e.g., Miglyol 812M), isopropylmyristate (oily) (e.g., kollicream IPM), oleoyl macrogol-6 glycerides (e.g., Labrafil 1944 cs), linoleoyl Polyoxyl-6 glycerides (e.g., Labrafil 2125 cs), and PEG-60 Hydrogenated Castor Oil (e.g., cremophor RH 60). In some embodiments, the nonionic emulsifier comprises glycerol monostearate. In some embodiments, the nonionic emulsifier comprises PEG stearate. In some embodiments, the nonionic emulsifier comprises glycerol monostearate and a PEG stearate. In some embodiments, the PEG stearate is PEG-75 stearate. In some embodiments, the PEG stearate is PEG stearate having a molecular weight of about 3-4 kDa. In some embodiments, the PEG stearate is PEG stearate having a molecular weight of about 3.5 kDa. In some embodiments, the nonionic emulsifier comprises isopropyl myristate (IPM).

In some embodiments, the nonionic emulsifier is present in the formulation at a concentration ranging from about 1-10% by weight. In some embodiments, the nonionic emulsifier is present in the formulation at a concentration ranging from about 1-2% by weight, 2-4% by weight, 4-6% by weight, 6-8% by weight, or 8-10% by weight. In some embodiments, the nonionic emulsifier is present in the formulation at a concentration of about 5% by weight. In some embodiments, the nonionic emulsifier is a combination of glycerol monostearate and a PEG stearate. In some embodiments, the PEG stearate has a molecular weight of about 3-4 kDa. In some embodiments, the PEG stearate has a molecular weight of about 3.5 kDa.

In some embodiments, the nonionic emulsifier is present in the formulation at a concentration ranging from about 1-2% by weight, 2-4% by weight, 4-6% by weight, 6-8% by weight, or 8-10% by weight. In some embodiments, the nonionic emulsifier is a combination of glycerol monostearate and a PEG stearate. In some embodiments, the PEG stearate has a molecular weight of about 3-4 kDa. In some embodiments, the PEG stearate has a molecular weight of about 3.5 kDa.

In some embodiments, the nonionic emulsifier is present in the formulation at a concentration of about 5% by weight. In some embodiments, the nonionic emulsifier is a combination of glycerol monostearate and a PEG stearate. In some embodiments, the PEG stearate has a molecular weight of about 3-4 kDa. In some embodiments, the PEG stearate has a molecular weight of about 3.5 kDa.

### 2. Emollients

Emollients are moisturizing and/or lubricating agents included in skin care products to help address dryness and irritation. For example, emollients can serve as a cream base in topical formulations.

In some embodiments, the formulation further comprises an emollient. Examples of emollients include but are not limited to cetostearyl alcohol (long chain alcohol) (e.g., Kolliwax® CSA 70), stearyl alcohol (long chain alcohol), myristyl stearate (long chain ester) (e.g., cetyl ester), cocoyl caprylocaprate (oily) (e.g., kollicream 3c), or polyethylene glycol (mw: 3350) (e.g., kollisolv PEG 3350). In some embodiments, the emollient comprises stearyl alcohol. In some embodiments, the emollient comprises cetyl alcohol or a combination of cetyl and stearyl alcohols. In some embodiments, the emollient is present in the formulation at a concentration ranging from about 1-10% by weight. In some embodiments, the nonionic emulsifier is present in the formulation at a concentration ranging from about 1-2% by weight, 2-4% by weight, 4-6% by weight, 5-7% by weight, 6-8% by weight, or 8-10% by weight. In some embodiments, the nonionic emulsifier is present in the formulation at a concentration of about 3% by weight. In some embodiments, the nonionic emulsifier is present in the formulation at a concentration of about 6% by weight. In some embodiments, the emollient is stearyl alcohol.

In some embodiments, the formulation further comprises an emollient. In some embodiments, the emollient comprises stearyl alcohol. In some embodiments, the emollient comprises cetyl alcohol or a combination of cetyl and stearyl alcohols. In some embodiments, the nonionic emulsifier is present in the formulation at a concentration of about 3% by weight. In some embodiments, the emollient is stearyl alcohol. In some embodiments, the nonionic emulsifier is present in the formulation at a concentration of about 6% by weight. In some embodiments, the emollient is stearyl alcohol.

### 3. Antioxidants

Inclusion of one or more antioxidants in a formulation can help limit oxidation and degradation of the active pharmaceutical ingredient and other ingredients. By mitigating excessive oxidation, antioxidants can protect the integrity of a formulation, supporting consistent efficacy and safety of the product, e.g., over time.

In some embodiments, the formulation further comprises an antioxidant. In some embodiments, the antioxidant comprises a thiosulfate. In some embodiments, the thiosulfate is sodium thiosulfate. In some embodiments, the antioxidant is present in the formulation at a concentration ranging from about 0.1-1% by weight. In some embodiments, the antioxidant is present in the formulation at a concentration ranging from about 0.1-1% by weight, wherein the antioxidant is sodium thiosulfate. In some embodiments, the antioxidant is present in the formulation at a concentration ranging from about 0.1-0.2% by weight, 0.2-0.4% by weight, 0.4-0.6% by weight, 0.6-0.8% by weight, or 0.8-1% by weight. In some embodiments, the antioxidant is present in the formulation at a concentration ranging from about 0.1-0.2% by weight, 0.2-0.4% by weight, 0.4-0.6% by weight, 0.6-0.8% by weight, or 0.8-1% by weight, wherein the antioxidant is sodium thiosulfate. In some embodiments, the antioxidant is present in the formulation at a concentration ranging from about 0.05-0.2% by weight or about 0.1% by weight, optionally wherein the antioxidant is sodium thiosulfate. In some embodiments, the antioxidant is present in the formulation at a concentration ranging from about 0.05-0.2% by weight or about 0.1% by weight, wherein the antioxidant is sodium thiosulfate.

In some embodiments, the formulation further comprises an antioxidant. In some embodiments, the antioxidant comprises a thiosulfate. In some embodiments, the thiosulfate is sodium thiosulfate. In some embodiments, the antioxidant is present in the formulation at a concentration ranging from about 0.05-0.2% by weight or about 0.1% by weight, optionally wherein the antioxidant is sodium thiosulfate. In some embodiments, the antioxidant is present in the formulation at a concentration ranging from about 0.05-0.2% by weight or about 0.1% by weight, wherein the antioxidant is sodium thiosulfate.

### 4. Preservatives

Preservatives are included in formulations to improve the shelf life of a formulation, e.g., by preventing microbial and/or fungal growth. Preservatives can help to mitigate degradation or decomposition of the active pharmaceutical ingredient and other ingredients and to support consistent efficacy and safety of the product, e.g., over time.

In some embodiments, the formulation further comprises a preservative. In some embodiments, the preservative comprises a benzoate. In some embodiments, the preservative is sodium benzoate. In some embodiments, the preservative is present in the formulation at a concentration ranging from about 0.1-1% by weight. In some embodiments, the preservative is present in the formulation at a concentration ranging from about 0.1-1% by weight, wherein the preservative is sodium benzoate. In some embodiments, the preservative is present in the formulation at a concentration ranging from about 0.05-0.15% by weight, 0.15-0.25% by weight, 0.25-0.35% by weight, or 0.35-0.5% by weight. In some embodiments, the preservative is present in the formulation at a concentration ranging from about 0.05-0.15% by weight, 0.15-0.25% by weight, 0.25-0.35% by weight, or 0.35-0.5% by weight, wherein the preservative is sodium benzoate. In some embodiments, the preservative is present in the formulation at a concentration ranging from about 0.15-0.25% by weight or about 0.2% by weight. In some embodiments, the preservative is present in the formulation at a concentration ranging from about 0.15-0.25% by weight or about 0.2% by weight, wherein the preservative is sodium benzoate.

In some embodiments, the formulation further comprises a preservative. In some embodiments, the preservative comprises a benzoate. In some embodiments, the preservative is sodium benzoate. In some embodiments, the preservative is present in the formulation at a concentration ranging from about 0.15-0.25% by weight or about 0.2% by weight. In some embodiments, the preservative is present in the formulation at a concentration ranging from about 0.15-0.25% by weight or about 0.2% by weight, wherein the preservative is sodium benzoate.

### 5. Excipients

Excipients are ingredients other than the active pharmaceutical ingredient (API) present in a finished pharmaceutical drug formulation, often used as lubricants, diluent, binders, flavorings, coating, and coloring agents.

In some embodiments, the formulation further comprises at least one additional excipient. In some embodiments, the at least one additional excipient comprises a PEG, optionally wherein the PEG is PEG400. In some embodiments, the at least one additional excipient comprises a PEG, wherein the PEG is PEG400. In some embodiments, the at least one additional excipient comprises a glycerol polyethylene glycol hydroxy stearate. In some embodiments, the at least one additional excipient comprises a hydrogenated ethoxylated castor oil. In some embodiments, the at least one additional excipient comprises a hydrogenated ethoxylated castor oil, wherein the hydrogenated ethoxylated castor oil is Peg-60 hydrogenated castor oil. In some embodiments, the at least one additional excipient comprises glycerin. In some embodiments, the at least one additional excipient is present in the formulation at a concentration ranging from about 0.1-0.2% by weight, 0.2-0.4% by weight, 0.4-0.6% by weight, 0.6-0.8% by weight, 0.8-1% by weight, 1-2% by weight, 2-4% by weight, 4-6% by weight, 5-7% by weight, 6-8% by weight, or 8-10% by weight.

### C. Formulations

Exemplary topical formulations include gels, hydrogels, lotions, solutions, creams, ointments, dusting powders, dressings, foams, films, skin patches, wafers, implants, sponges, fibers, bandages, and emulsions. Exemplary excipients include alcohol, water, mineral oil, liquid petrolatum, white petrolatum, glycerin, polyethylene glycol, polypropylene glycol (PPG), propylene glycol, and diethylene glycol monoethyl ether.

In some embodiments, phenylbutyrate is incorporated into a cream, gel, lotion, ointment, foam, suppository, or a spray. Methods for preparation of these types of topical formulations are known in the art. Suitable excipients include emulsifiers, organogelators and emollients. Emulsifiers include polyethylene glycol stearate, a glycol stearate, a glyceryl stearate, cetearyl alcohol and ceteareth 20, methylcellulose, Cetomacrogol 1000, lecithin, PEG-8-beeswax (e.g., APIFIL), glycerol monostearate/PEG75 stearate (e.g., Gelot 64), PEG-6-stearate/ethylene glycol stearates/PEG75 stearate (e.g., Tefose 63), medium chain triglycerides (C8/C10 triglyceride) (e.g., Miglyol 812M), isopropylmyristate (oily) (e.g., kollicream IPM), oleoyl macrogol-6 glycerides (e.g., Labrafil 1944 cs), linoleoyl Polyoxyl-6 glycerides (e.g., Labrafil 2125 cs), and Peg-60 Hydrogenated Castor Oil (e.g., cremophor RH 60). Suitable organogelators include 4-tertbutyl-1-aryl cyclohexanols derivatives, polymeric (e.g., poly(ethylene glycol), polycarbonate, polyesters, and poly(alkylene), Gemini gelators (e.g., N-lauroyl-L-lysine ethyl ester), Boc-Ala (1)-Aib (2)-β-Ala (3)-OMe (synthetic tripeptide), and low molecular weight gelators (e.g., fatty acids and n-alkanes). Suitable emollients include cetostearyl alcohol, cetyl alcohol, isopropyl palmitate, caprylic/capric triglyceride, PPG-2 myristyl ether propionate, dimethicone, methicone, petrolatum, lanolin, and mineral oil.

If desired, other additives including surfactants, penetration enhancers, preservatives, viscosity modifiers, and emulsion stabilizers may be included in the formulations. Suitable surfactants include sodium lauryl sulfate, cetostearyl alcohol, ceteareth 12, ceteareth 20, cetearyl alcohol, Cetomacrogol 1000, stearic acid, poloxamer, polyethylene glycol hexadecyl ether (C16 PED-20) (e.g., Brij 58), polysorbate 80 (e.g., Tween 80), oleoyl macrogol glycerides (e.g., Labrafil M 1944 CS), caprylocaproyl macrogol-8-glycerides (e.g., Labrasol), lauroyl polyoxyl-32 glycerides (microemulsion) (e.g., Gelucire 44/14), stearoyl polyoxyl-32 glycerides (e.g., Gelucire 50/13), and propylene glycol monocaprylate (e.g., Lauryl glycol FCC). Suitable penetration enhancers include propylene glycol and carboxypolymethylene (e.g., Carbomer 974P). Suitable preservatives include methylparaben, propylparaben, ethylhexylglycerin, phenoxyethanol, chlorocresol, potassium sorbate, sorbic acid, bronopol, methychloroisothiazolinone, and methylisothiazolinone. Suitable viscosity modifiers include carboxymethylcellulose, carboxyethylcellulose, acrylate crosspolymer, and carbomer. Suitable emulsion stabilizers include xanthan gum, glyceryl stearate, and carbomer. Suitable additional hydrophilic components include glycerin, propylene glycol, transcutol p (e.g., diethylene glycol monoethyl ether), and PEG 400 (e.g., polyethylene glycol (mw: 400)). If desired, other additives may be included to modify the color or aroma of the topical compositions described herein.

Transdermal delivery of drugs through the skin is another recognized method for maintaining relatively steady plasma levels of therapeutic agents whilst also circumventing the pain, discomfort and inconvenience of intravenous, oral and rectal drug delivery.

Provided herein is a pharmaceutical formulation comprising an optionally 4-substituted phenylbutyrate in an emulsion, wherein the formulation is a topical formulation. In some embodiments, the pharmaceutical formulations comprising an optionally 4-substituted phenylbutyrate in an emulsion, wherein the phenylbutyrate is present at a concentration ranging from about 1-40% by weight. In some embodiments, the phenylbutyrate is present at a concentration ranging from about 2-40% by weight. In some embodiments, provided herein is a pharmaceutical formulation comprising an optionally 4-substituted phenylbutyrate in an emulsion, wherein the emulsion comprises a hydrophobic phase and an aqueous phase, and wherein the formulation is a topical formulation. In some embodiments, the aqueous phase comprises water and one or more additional hydrophilic components, and the one or more additional hydrophilic components are present in a ratio of 1:1 or greater by weight relative to the optionally 4-substituted phenylbutyrate. In some embodiments, the hydrophobic phase is dispersed in the aqueous phase.

In some embodiments, the formulation is a topical formulation, wherein the topical formulation is a cream, ointment, gel, spray, or foam. In some embodiments, the topical formulation is a cream. In some embodiments, the topical formulation is ointment. In some embodiments, the topical formulation is gel. In some embodiments, the topical formulation is spray. In some embodiments, the topical formulation is foam. In some embodiments, the topical formulation is contained in a patch or dressing. In some embodiments, the patch is an adhesive patch. In some embodiments, the dressing is a non-adhesive dressing. In some embodiments, the dressing is an occlusive dressing. In some embodiments, the dressing is a non-occlusive dressing.

In some embodiments, the formulation comprises less than about 0.01%, 0.001%, 0.0005%, or 0.0003% by weight of related compound A and salts thereof. In some embodiments, the formulation comprises less than about 0.01% by weight of related compound A and salts thereof. In some embodiments, the formulation comprises less than about 0.001% by weight of related compound A and salts thereof. In some embodiments, the formulation comprises less than about 0.0005% by weight of related compound A and salts thereof. In some embodiments, the formulation comprises less than about 0.0003% by weight of related compound A and salts thereof. Related compound A comprises the structure as shown below.

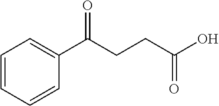 [SMILES: O=C(O)CCC(=O)c1ccccc1]

(related compound A)

In some embodiments, the formulation undergoes less than about 0.01%, 0.001%, 0.0005%, or 0.0003% conversion by weight of the phenylbutyrate to related compound A and salts thereof upon storage at 60° C. for 21 days. In some embodiments, the formulation undergoes less than about 0.01% conversion by weight of the phenylbutyrate to related compound A and salts thereof upon storage at 60° C. for 21 days. In some embodiments, the formulation undergoes less than about 0.001% conversion by weight of the phenylbutyrate to related compound A and salts thereof upon storage at 60° C. for 21 days. In some embodiments, the formulation undergoes less than about 0.0005% conversion by weight of the phenylbutyrate to related compound A and salts thereof upon storage at 60° C. for 21 days.

In some embodiments, the formulation undergoes less than about 0.01%, 0.001%, 0.0005%, or 0.0003% conversion by weight of the phenylbutyrate to related compound A and salts thereof upon storage at 25±2° C. and 60±5% relative humidity (RH) for at least 1 month, 3 months, 6 months, or 12 months. In some embodiments, the formulation undergoes less than about 0.01%, 0.001%, 0.0005%, or 0.0003% conversion by weight of the phenylbutyrate to related compound A and salts thereof upon storage at 40±2° C. and 75±5% RH for at least 1 month, 3 months, or 6 months.

In some embodiments, the formulation comprises less than about 0.5%, 0.05%, or 0.005% by weight of total impurities upon storage at 25±2° C. and 60±5% relative humidity (RH) for at least 1 month, 3 months, 6 months, or 12 months. In some embodiments, the formulation comprises less than about 0.5%, 0.05%, or 0.005% by weight of total impurities upon storage at 40±2° C. and 75±5% RH for at least 1 month, 3 months, or 6 months. In some embodiments, the formulation comprises less than about 0.5%, 0.05%, or 0.005% by weight of total impurities upon storage at 60° C. for 7 days or 21 days.

In some embodiments, the formulation comprises a buffer. In some embodiments, the formulation has a pH of about 4, 5, 6, 7, or 8. In some embodiments, the formulation has a pH in the range of about 4-8, 5-7, or 6.2-7.2. In some embodiments, the formulation has a pH in the range of about 6.5-7.

In some embodiments, the formulation has a viscosity of about 3500, 4000, 4500, 5000, 5500, 6000, or 6500 cps. In some embodiments, the formulation has a viscosity in the range of about 3500-6500 cps. In some embodiments, the formulation has a viscosity in the range of about 4500-5500 cps.

In some embodiments, the formulation has a microbial limit. In some embodiments, the formulation has one or more microbial limits. In some embodiments, the microbial limit comprises a negative test for Escherichia coli test. In some embodiments, the microbial limit comprises a total yeast and mold count (TYMC) that is not more than 100 colony forming units (cfu)/mL. In some embodiments, the microbial limit comprises a total aerobic microbial count (TAMC) that is not more than 1000 cfu/mL. In some embodiments, the microbial limit comprises a negative test for Staphylococcus aureus.

In some embodiments, the pharmaceutical formulation comprises 10% sodium phenylbutyrate, 5% Gelot™ 64 (Mono and Diglycerides and Polyoxyl stearate), 6% Kolliwax® CSA 70 (cetostearyl alcohol), 6% glycerin, 0.5% sodium thiosulfate (20% solution), 0.2% sodium benzoate, and 72.3% water. In some embodiments, the pharmaceutical formulation consists of 10% sodium phenylbutyrate, 5% Gelot™ 64, 6% Kolliwax® CSA 70, 6% glycerin, 0.5% sodium thiosulfate (20% solution), 0.2% sodium benzoate, and 72.3% water. Formulation ingredients expressed using a percentage are the percentage by weight of the formulation.

In some embodiments, the pharmaceutical formulation comprises 10% sodium phenylbutyrate, 5% Gelot™ 64 (Mono and Diglycerides and Polyoxyl stearate), 10% Kolliwax® CSA 70 (cetostearyl alcohol), 6% glycerin, 0.5% sodium thiosulfate (20% solution), 0.2% sodium benzoate, and 72.3% water. In some embodiments, the pharmaceutical formulation consists of 10% sodium phenylbutyrate, 5% Gelot™ 64, 6% Kolliwax® CSA 70, 6% glycerin, 0.5% sodium thiosulfate (20% solution), 0.2% sodium benzoate, and 68.3% water. Formulation ingredients expressed using a percentage are the percentage by weight of the formulation.

In some embodiments, the pharmaceutical formulation comprises 10% sodium phenylbutyrate, 5% Gelot™ 64 (Mono and Diglycerides and Polyoxyl stearate), 10% stearyl alcohol, 6% glycerin, 0.5% sodium thiosulfate (20% solution), 0.2% sodium benzoate, and 72.3% water. In some embodiments, the pharmaceutical formulation consists of 10% sodium phenylbutyrate, 5% Gelot™ 64, 6% Kolliwax® stearyl alcohol, 6% glycerin, 0.5% sodium thiosulfate (20% solution), 0.2% sodium benzoate, and 68.3% water. Formulation ingredients expressed using a percentage are the percentage by weight of the formulation.

In some embodiments, the pharmaceutical formulation comprises 10% sodium phenylbutyrate, 5% Gelot™ 64, 3% Kolliwax® CSA 70, 7% PEG 400, 5% isopropyl myristate (IPM), 1% RH60, 8% glycerin, 0.5% sodium thiosulfate (20% solution), 0.2% sodium benzoate, and 60.3% water. In some embodiments, the pharmaceutical formulation consists of 10% sodium phenylbutyrate, 5% Gelot™ 64, 3% Kolliwax® CSA 70, 7% PEG 400, 5% isopropyl myristate (IPM), 1% RH60, 8% glycerin, 0.5% sodium thiosulfate (20% solution), 0.2% sodium benzoate, and 60.3% water. Formulation ingredients expressed using a percentage are the percentage by weight of the formulation.

In some embodiments, the pharmaceutical formulation comprises 10% sodium phenylbutyrate, 5% Gelot™ 64, 6% Kolliwax® CSA 70, 0.5% Carbomer 974P, 1% RH40, 5% glycerin, 0.5% sodium thiosulfate (20% solution), 0.2% sodium benzoate, and 71.8% water. In some embodiments, the pharmaceutical formulation consists of 10% sodium phenylbutyrate, 5% Gelot™ 64, 6% Kolliwax® CSA 70, 0.5% Carbomer 974P, 1% RH40, 5% glycerin, 0.5% sodium thiosulfate (20% solution), 0.2% sodium benzoate, and 71.8% water. Formulation ingredients expressed using a percentage are the percentage by weight of the formulation.

### 1. In Vitro Release Properties

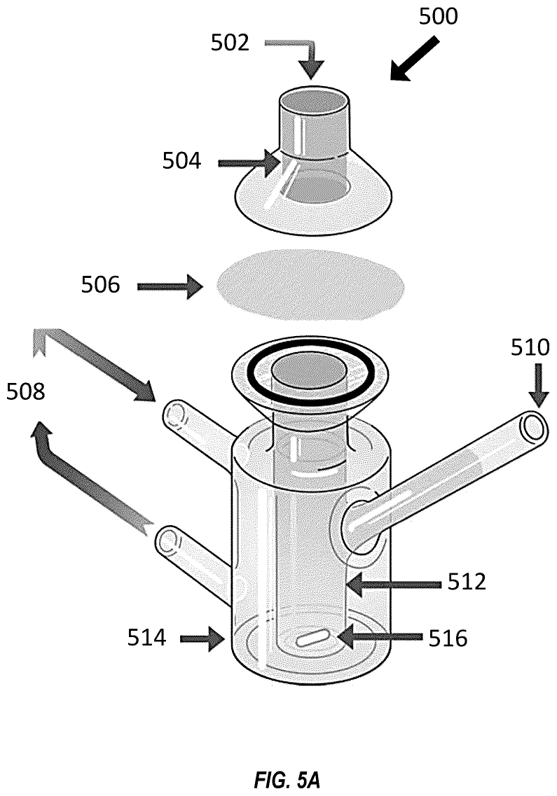

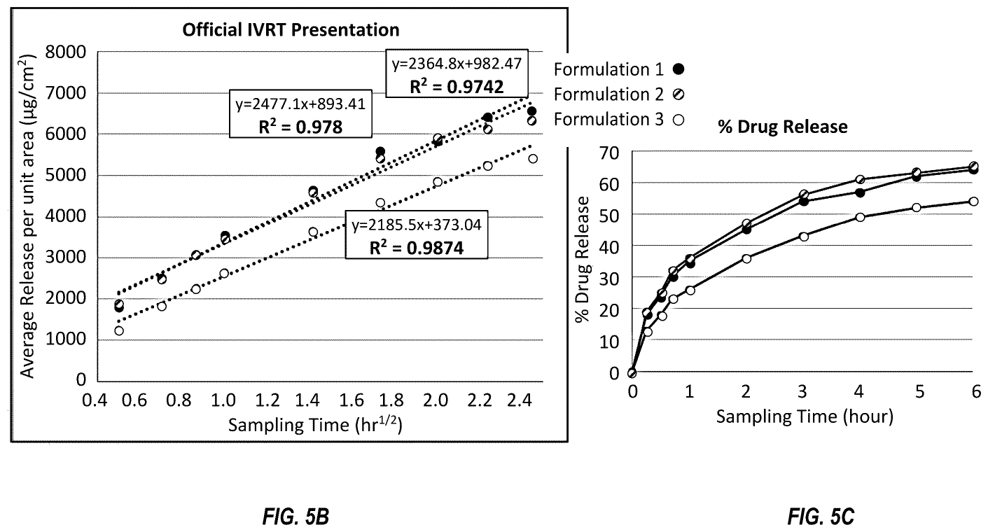

Described herein is an in vitro release test (IVRT) that is useful for assessing the performance of any one of the pharmaceutical formulations described herein (e.g., formulation drug release). The modified Franz Vertical Diffusion Cell apparatus 500 for conducting the IVRT is shown in FIG. 5A. Once warmed to the appropriate temperature and the formulation sample has been added (e.g., about 32° C.), the synthetic nylon membrane 506 containing sample is placed on top of the receptor chamber 512. The donor chamber is lowered to contact the nylon membrane, and the donor chamber 504 and the receptor chamber are then clamped together to secure the nylon membrane. Additional test compounds can be added to the nylon membrane through the donor compound opening 502. In the receptor chamber, sampling buffer can be added through the sampling port 510 and mixed using an added stir bar 516. The receptor chamber is housed within a water jacket 514 that is connected to a circulating heating source 508.

Provided herein is a pharmaceutical formulation comprising an optionally 4-substituted phenylbutyrate in an emulsion, wherein the emulsion comprises a hydrophobic phase and an aqueous phase, wherein the aqueous phase comprises water and one or more additional hydrophilic components, wherein the formulation has the property of at least about 40% phenylbutyrate release at 6 hours under in vitro release testing conditions, and wherein the formulation is a topical formulation. In some embodiments, the formulation has the property of at least about 50%, 60%, or 70% phenylbutyrate release at 6 hours under in vitro release testing conditions. In some embodiments, the formulation has the property of at least about 40%, 50%, 60%, or 70% phenylbutyrate release at 5 hours under in vitro release testing conditions. In some embodiments, the formulation has the property of at least about 40%, 50%, 60%, or 70% phenylbutyrate release at 4 hours under in vitro release testing conditions. In some embodiments, the formulation has the property of at least about 40%, 50%, 60%, or 70% phenylbutyrate release at 3 hours under in vitro release testing conditions. In some embodiments, the formulation has the property of at least about 40%, 50%, or 60% phenylbutyrate release at 2 hours under in vitro release testing conditions. In some embodiments, the formulation has the property of at least about 70% phenylbutyrate release at 2 hours under in vitro release testing conditions.

### 2. Skin Retention and Permeation Properties

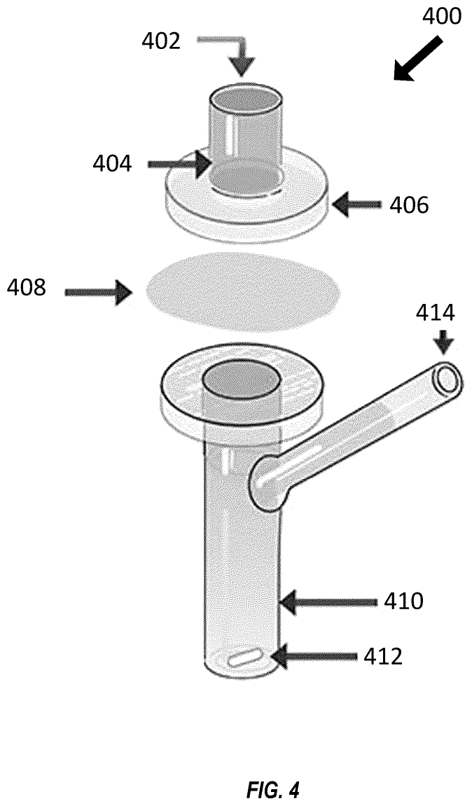

Described herein is an in vitro skin retention and permeation assay that is useful for assessing the performance of any one of the pharmaceutical formulations described herein (e.g., formulation skin retention). The modified Franz cell system 400 for conducting the in vitro skin retention and permeation assay is shown in FIG. 4. Once prepared, the cadaver skin sample 408 is placed on the donor chamber 404. The donor chamber and the receptor chamber 410 are then clamped together using the flat flange joint 406 to secure the cadaver skin sample. Test compound can then be added to the cadaver skin sample through the donor compound opening 402. In the receptor chamber, sampling buffer (e.g., phosphate buffer) can be added through the sampling port 414 and mixed using an added stir bar 412. Not shown is the release liner which can be added to the applied sample to ensure even distribution of the formulation across the cadaver skin sample.

Provided herein is a pharmaceutical formulation comprising an optionally 4-substituted phenylbutyrate in an emulsion, wherein the emulsion comprises a hydrophobic phase and an aqueous phase, wherein the aqueous phase comprises water and one or more additional hydrophilic components, wherein the formulation has the property of at least about 5% phenylbutyrate skin retention at 6 hours under in vitro skin retention assay conditions, and wherein the formulation is a topical formulation. In some embodiments, the formulation has the property at least about 10%, 15%, or 20% phenylbutyrate skin retention at 6 hours under in vitro skin retention assay conditions. In some embodiments, the formulation has the property at least about 15%, 16%, 17%, 18%, 19%, or 20% phenylbutyrate skin retention at 6 hours under in vitro skin retention assay conditions. In some embodiments, the formulation has the property at least about 15% phenylbutyrate skin retention at 6 hours under in vitro skin retention assay conditions.

Also provided herein is a pharmaceutical formulation comprising an optionally 4-substituted phenylbutyrate in an emulsion, wherein the emulsion comprises a hydrophobic phase and an aqueous phase, wherein the aqueous phase comprises water and one or more additional hydrophilic components, wherein the formulation has the property of no more than about 12% phenylbutyrate skin permeation at 6 hours under in vitro skin retention assay conditions, and wherein the formulation is a topical formulation. In some embodiments, the formulation has the property of no more than about 10%, 8%, 6%, or 4% phenylbutyrate skin permeation at 6 hours under in vitro skin retention assay conditions. In some embodiments, the formulation has the property of no more than about 10%, 9%, 8%, 7%, 6%, 5%, or 4% phenylbutyrate skin permeation at 6 hours under in vitro skin retention assay conditions. In some embodiments, the formulation has the property of no more than about 4% phenylbutyrate skin permeation at 6 hours under in vitro skin retention assay conditions.

Also provided herein is a pharmaceutical formulation comprising an optionally 4-substituted phenylbutyrate in an emulsion, wherein the emulsion comprises a hydrophobic phase and an aqueous phase, wherein the aqueous phase comprises water and one or more additional hydrophilic components, wherein the formulation has the property of at least about 1:1 ratio of phenylbutyrate skin retention to skin permeation at 6 hours under in vitro skin retention assay conditions, and wherein the formulation is a topical formulation. In some embodiments, the formulation has the property of at least about 2:1, 3:1, or 4:1 ratio of phenylbutyrate skin retention to skin permeation at 6 hours under in vitro skin retention assay conditions. In some embodiments, the formulation has the property of at least about 4:1 ratio of phenylbutyrate skin retention to skin permeation at 6 hours under in vitro skin retention assay conditions.

### 3. Formulation Stability

Assessing the stability of a formulation involves storage under stress conditions (e.g., increased heat, increased humidity) for extended periods (e.g., days, weeks, or months) to assess changes in the formulation composition and integrity. This includes changes in active pharmaceutical ingredient composition or abundance, impurities, as well as other features and characteristics important for formulation, use, safety, and efficacy. For example, a topical formulation can be assessed for assay performance, analyte and metabolite abundance, visual changes, viscosity, and pH after stress condition storage.

In some embodiments, the formulation is stable under stress storage conditions comprising a temperature of at least 25±2° C. and a relative humidity (RH) at least 60±5% for at least 1, 2, 3, 4, 6, 8, 10, or 12 weeks. In some embodiments, the formulation is stable under stress storage conditions comprising a temperature of at least 25±2° C. and a relative humidity (RH) at least 60±5% for at least 1, 3, 6, or 12 months. In some embodiments, the formulation is stable under stress storage conditions comprising a temperature of at least 25±2° C. and a relative humidity (RH) at least 60±5% for at least 6 or 12 months.

In some embodiments, the formulation is stable under stress storage conditions comprising a temperature of at least 40±2° C. and a relative humidity (RH) at least 75±5% for at least 1, 2, 3, 4, 6, 8, 10, or 12 weeks. In some embodiments, the formulation is stable under stress storage conditions comprising a temperature of at least 40±2° C. and a relative humidity (RH) at least 75±5% for at least 1, 3, or 6 months. In some embodiments, the formulation is stable under stress storage conditions comprising a temperature of at least 40±2° C. and a relative humidity (RH) at least 75=5% for at least 6 months.

In some embodiments, the formulation is stable under stress storage conditions comprising a temperature of at least 60° C. for at least 7, 14, or 21 days. In some embodiments, the formulation is stable under stress storage conditions comprising a temperature of at least 60° C. for at least 21 days.

### III. Methods of Preparing

Provided herein are methods of preparing the formulation comprising emulsifying and homogenizing a hydrophobic phase and an aqueous phase to produce said formulation. In some embodiments, the formulation comprises an optionally 4-substituted phenylbutyrate in an emulsion, wherein the phenylbutyrate is present at a concentration ranging from about 1-40% by weight, wherein the emulsion comprises a hydrophobic phase dispersed in an aqueous phase, and wherein the formulation is a topical formulation. In some embodiments, the phenylbutyrate is present at a concentration ranging from about 2-40% by weight. In some embodiments, the methods of preparing provided herein can comprise producing any of the formulations described elsewhere herein, including but not limited to Section II, the numbered embodiments, and the Examples.

In some embodiments, the method of preparing a formulation comprises adding sterilized water and sodium phenylbutyrate together and mixing. In some embodiments, the method comprises further adding glycerol, sodium thiosulfate solution 20% and sodium benzoate and mixing. In some embodiments, the method comprises further adding mono and diglycerides, polyoxyl stearate (e.g., Gelot™ 64) and cetostearyl alcohol (e.g., Kolliwax® CSA 50) and mixing for 60 minutes at 80° C. In some embodiments, the method comprises further mixing the mixture for 90 minutes at 60° C. In some embodiments, the method further comprises allowing the mixture to cool to room temperature. In some embodiments, the method further comprises using a peristaltic pump for the filling process, wherein the topical formulation is heated to 60° C. and mixed, and then filled into the package material with pumping speed at 30±0.5 rpm.

In some embodiments, the method of preparing a formulation comprises adding 683 g sterilized water and 100 g sodium phenylbutyrate together and mixing for 5 minutes at 150 rpm. In some embodiments, the method comprises further adding 60 g glycerol, 5 g sodium thiosulfate solution 20% and 2 g sodium benzoate and mixing for 5 minutes at 150 rpm. In some embodiments, the method comprises further adding 50 g mono and diglycerides, polyoxyl stearate (Gelot™ 64) and 100 g cetostearyl alcohol (Kolliwax® CSA 50) and mixing, e.g., at 150±50 rpm, such as about 180 rpm, for 60 minutes at 80° C. In some embodiments, the method comprises further mixing the mixture at 400 rpm for 90 minutes at 60° C. In some embodiments, the method further comprises allowing the mixture to cool to room temperature, e.g., for 2-3 hours. In some embodiments, the method further comprises using a peristaltic pump for the filling process, wherein the topical formulation is heated to 60° C. and mixed at 400 rpm, and then filled into the package material with pumping speed at 30±0.5 rpm.

In some embodiments, the method of preparing a formulation comprises adding sterilized water and sodium phenylbutyrate together and mixing. In some embodiments, the method comprises further adding glycerol, sodium thiosulfate solution 20% and sodium benzoate and mixing. In some embodiments, the method comprises further adding mono and diglycerides, polyoxyl stearate (Gelot™ 64) and stearyl alcohol and mixing for 60 minutes at 80° C. In some embodiments, the method comprises further mixing the mixture for 90 minutes at 60° C. In some embodiments, the method further comprises allowing the mixture to cool to room temperature. In some embodiments, the method further comprises using a peristaltic pump for the filling process, wherein the topical formulation is heated to 60° C. and mixed, and then filled into the package material with pumping speed at 30±0.5 rpm.

In some embodiments, the method of preparing a formulation comprises adding 683 g sterilized water and 100 g sodium phenylbutyrate together and mixing for 5 minutes at 150 rpm. In some embodiments, the method comprises further adding 60 g glycerol, 5 g sodium thiosulfate solution 20% and 2 g sodium benzoate and mixing for 5 minutes at 150 rpm. In some embodiments, the method comprises further adding 50 g mono and diglycerides, polyoxyl stearate (Gelot™ 64) and 100 g stearyl alcohol and mixing, e.g., at 150±50 rpm, such as about 180 rpm for 60 minutes at 80° C. In some embodiments, the method comprises further mixing the mixture at 400 rpm for 90 minutes at 60° C. In some embodiments, the method further comprises allowing the mixture to cool to room temperature, e.g., for 2-3 hours. In some embodiments, the method further comprises using a peristaltic pump for the filling process, wherein the topical formulation is heated to 60° C. and mixed at 400 rpm, and then filled into the package material with pumping speed at 30=0.5 rpm.

In some embodiments, the method further comprises validating the prepared formulation completing one or more of a visual inspection, an active pharmaceutical ingredient (API) identification, a content uniformity assessment, a skin retention assay, an impurity analysis, a pH assessment, a viscosity assessment, an in vitro release test, a microbial limit analysis, and a preservative analysis. In some embodiments, the method further comprises validating the prepared formulation completing at least 1, 2, 3, 4, 5, 6, 7, 8, 9, or all of the assessments consisting of a visual inspection, an active pharmaceutical ingredient (API) identification, a content uniformity assessment, a skin retention assay, an impurity analysis, a pH assessment, a viscosity assessment, an in vitro release test, a microbial limit analysis, and a preservative analysis.

### IV. Uses and Methods of Treatment

Described in this section are methods of treatment, comprising applying the formulation of any one of those described herein topically to a subject in need thereof. Methods of delivering a phenylbutyrate to the skin of a subject are also described, comprising applying the formulation of any one of those described herein topically to the subject. Uses of the formulation of any one of those described here for the manufacture of a medicament, wherein the medicament is for treating skin inflammation (e.g., psoriasis).

Provided herein are methods of treating skin inflammation, comprising applying the formulation of any one of those described herein (e.g., Section II) topically to a subject in need thereof. In some instances, provided herein is a method of treating psoriasis, comprising applying the formulation of any one of those described herein (e.g., Section II) topically to a subject having psoriasis. In some instances, provided herein is a method of treating a skin condition, comprising applying the formulation of any one of those described herein (e.g., Section II) topically to a subject having the skin condition.

Without being bound to theory, phenylbutyrate (PB) may have novel mechanisms of action not previously recognized and/or fully appreciated. PB may act as a chemical chaperone to reduce endoplasmic reticulum (ER) stress and the unfolded protein response (UPR), which are implicated in a broad spectrum of skin disorders, including psoriasis. PB may also target TAS2R14 (bitter taste receptor agonist), which is highly expressed in skin, to help restore the cholecystokinin (CCK) pathway critical for normal skin function—a pathway known to be impaired in psoriasis and other inflammatory skin disorders. These biological activities could lead to (1) induction of keratinocyte differentiation and restored epidermal barrier, (2) inhibition of Th1/Th2 inflammatory cytokines (e.g., TNF-α, IL-1β and IL-6, IL-17A, IL-23; mediated by inhibition of NFκB-p65 activation and TLR4), and (3) anti-microbial activity against bacteria (e.g., Staphylococcus aureus, methicillin resistant), fungi (e.g., Candida albicans), or viruses (e.g., herpes simplex virus) associated with skin disorders and diseases. Herein, uses and methods of treatment are provided comprising the formulation of any one of those described above (e.g., in Section II).

In some instances, provided herein is a method of delivering a phenylbutyrate to the skin of a subject, comprising applying the formulation of any one of those described herein (e.g., Section II) topically to the subject.

Also provided herein is a use of the formulation of any one of those described herein (e.g., Section II) for the manufacture of a medicament. In some instances, provided herein is a use of the formulation of any one of those described herein (e.g., Section II) for the manufacture of a medicament for treating skin inflammation. In some instances, provided herein is a use of the formulation of any one of those described herein (e.g., Section II) for the manufacture of a medicament for treating psoriasis. In some instances, provided herein is a use of the formulation of any one of those described herein (e.g., Section II) for the manufacture of a medicament for treating a skin condition.

Also provided herein is the formulation of any one of those described herein (e.g., Section II) for use in therapy. In some instances, provided herein is the formulation of any one of those described herein (e.g., Section II) for use in treating skin inflammation. In some instances, provided herein is the formulation of any one of those described herein (e.g., Section II) for use in treating psoriasis. In some instances, provided herein is the formulation of any one of those described herein (e.g., Section II) for use in treating a skin condition.

In some embodiments, the methods, uses, or formulations for use described herein are for treating a skin condition. In some embodiments, the skin condition includes skin inflammation or an inflammatory skin disorder. In some embodiments, the methods, uses, or formulations for use described herein are for treating an inflammatory skin disorder, psoriasis, epidermolysis bullosa, epidermolysis bullosa simplex, atopic dermatitis, eczema, seborrheic dermatitis, rosacea, toxic epidermal necrolysis, Stevens Johnson syndrome, Lyell syndrome, erythema multiforme, Necrobiosis lipoidica, Peeling skin syndrome, Ichthyosis, Neurodermatitis, Pemphigus, folliculitis, skin inflammation caused by a cancer therapy optionally wherein the cancer therapy comprises an epidermal growth factor inhibitor, skin cancer, melanoma, squamous cell carcinoma, basal cell carcinoma, Merkel cell carcinoma, dermatofibrosarcoma protuberans, actinic keratosis, sebaceous carcinoma, a skin wound, a skin ulcer, a venous skin ulcer, a diabetic skin ulcer, arthritis, osteoarthritis, psoriatic arthritis, itching, pruritus, cholestatic pruritus, skin aging, skin wrinkles, a gram-positive bacterial skin infection, a staphylococcal skin infection, an S. aureus skin infection, acne, and/or advanced glycation end-product accumulation. In some embodiments, the methods, uses, or formulations for use described herein are for treating for treating a subject having one or more of the said indications.

In some embodiments, the formulation of any one of those described herein (e.g., Section II) is administered to a subject in need thereof once or more daily. In some embodiments, the formulation of any one of those described herein (e.g., Section II) is administered to a subject in need thereof once daily. In some embodiments, the formulation of any one of those described herein (e.g., Section II) is administered to a subject in need thereof twice daily. In some embodiments, the formulation of any one of those described herein (e.g., Section II) is administered to a subject in need thereof as needed.

In some embodiments, treatment according to the methods, uses, and formulations for use provided herein results in improved skin health comprising reduced skin redness, smaller skin lesions, and reduced skin itching. In some embodiments, the method of treatment results in improved skin health in about 2-14 days, 4-12 days, or 6-10 days. In some embodiments, the method of treating further comprises applying or delivering said formulation to the subject in need twice daily.

### A. Indications and Patient Populations

Disclosed methods, uses, or formulations for use herein comprise treating an inflammatory skin disorder in a subject in need thereof. The subject may have one or more indication that may benefit from the application of any one of the formulations described herein.

In some embodiments, the subject receiving treatment has one or more of an inflammatory skin disorder, psoriasis, epidermolysis bullosa, epidermolysis bullosa simplex, atopic dermatitis, eczema, seborrheic dermatitis, rosacea, toxic epidermal necrolysis, Stevens Johnson syndrome, Lyell syndrome, erythema multiforme, Necrobiosis lipoidica, Peeling skin syndrome, Ichthyosis, Neurodermatitis, Pemphigus, folliculitis, skin inflammation caused by a cancer therapy optionally wherein the cancer therapy comprises an epidermal growth factor inhibitor, skin cancer (Eigner et al., Sci Rep 2017 Dec. 13; 7 (1): 17498), melanoma, squamous cell carcinoma, basal cell carcinoma, Merkel cell carcinoma, dermatofibrosarcoma protuberans, actinic keratosis, sebaceous carcinoma, a skin wound (Burian et al., Int J Mol Sci. 2022 Jun. 10; 23 (12): 6526), a skin ulcer (Bachar-Wikstrom et al., Int Wound J 2021 February; 18 (1): 49-61), a venous skin ulcer, a diabetic skin ulcer, arthritis and/or osteoarthritis (Tang et al., Biotechnol Appl Biochem. 2018 July; 65 (4): 540-546), psoriatic arthritis, itching (Hasegawa et al., Orphanet J Rare Dis 2014, 9:89), pruritus, cholestatic pruritus, skin aging (Ono K. and Nakashima M., Biomed Rep 2020 13 (6) 61), skin wrinkles, a gram-positive bacterial skin infection, a staphylococcal skin infection, an S. aureus skin infection (Wang et al., Molecules 2018 Nov. 22; 23 (12): 3056; Singh et al., Infect Immun 2023 Jan. 24; 91 (1): e0025222), acne, and/or advanced glycation end-product accumulation.

In some embodiments, the subject receiving treatment has an inflammatory skin disorder. In some embodiments, the subject receiving treatment has psoriasis. In some embodiments, the subject receiving treatment has epidermolysis bullosa. In some embodiments, the subject receiving treatment has epidermolysis bullosa simplex. In some embodiments, the subject receiving treatment has atopic dermatitis. In some embodiments, the subject receiving treatment has eczema. In some embodiments, the subject receiving treatment has seborrheic dermatitis. In some embodiments, the subject receiving treatment has rosacea. In some embodiments, the subject receiving treatment has toxic epidermal necrolysis. In some embodiments, the subject receiving treatment has Stevens Johnson syndrome. In some embodiments, the subject receiving treatment has Lyell syndrome. In some embodiments, the subject receiving treatment has erythema multiforme. In some embodiments, the subject receiving treatment has Necrobiosis lipoidica. In some embodiments, the subject receiving treatment has Peeling skin syndrome. In some embodiments, the subject receiving treatment has Ichthyosis. In some embodiments, the subject receiving treatment has Neurodermatitis. In some embodiments, the subject receiving treatment has Pemphigus. In some embodiments, the subject receiving treatment has folliculitis.

In some embodiments, the subject receiving treatment has skin inflammation caused by a cancer therapy. In some embodiments, the cancer therapy comprises an epidermal growth factor inhibitor. In some embodiments, the subject receiving treatment has a skin cancer (Eigner et al., Sci Rep 2017 Dec. 13; 7 (1): 17498). In some embodiments, the skin cancer is selected from the group consisting of melanoma, squamous cell carcinoma, basal cell carcinoma, Merkel cell carcinoma, dermatofibrosarcoma protuberans, and sebaceous carcinoma. In some embodiments, the subject receiving treatment has melanoma. In some embodiments, the subject receiving treatment has squamous cell carcinoma. In some embodiments, the subject receiving treatment has basal cell carcinoma. In some embodiments, the subject receiving treatment has Merkel cell carcinoma. In some embodiments, the subject receiving treatment has dermatofibrosarcoma protuberans. In some embodiments, the subject receiving treatment has sebaceous carcinoma. In some embodiments, the subject receiving treatment is at risk of developing skin cancer. In some embodiments, the subject receiving treatment has actinic keratosis.

In some embodiments, the subject receiving treatment has a skin wound (Burian et al., Int J Mol Sci. 2022 Jun. 10; 23 (12): 6526). In some embodiments the skin would is a skin ulcer (Bachar-Wikstrom et al., Int Wound J 2021 February; 18 (1): 49-61), a venous skin ulcer, and/or a diabetic skin ulcer. In some embodiments, the subject receiving treatment has skin ulcer. In some embodiments, the subject receiving treatment has a venous skin ulcer. In some embodiments, the subject receiving treatment has a diabetic skin ulcer.

In some embodiments, the subject receiving treatment has arthritis (Tang et al., Biotechnol Appl Biochem. 2018 July; 65 (4): 540-546). In some embodiments, the subject receiving treatment has osteoarthritis (Tang et al., Biotechnol Appl Biochem. 2018 July; 65 (4): 540-546). In some embodiments, the subject receiving treatment has psoriatic arthritis. In some embodiments, the subject receiving treatment has itching (Hasegawa et al., Orphanet J Rare Dis 2014, 9:89). In some embodiments, the subject receiving treatment has pruritus. In some embodiments, the subject receiving treatment has cholestatic pruritus.

In some embodiments, the subject receiving treatment has skin aging (Ono K. and Nakashima M., Biomed Rep 2020 13 (6) 61). In some embodiments, the subject receiving treatment has skin wrinkles. In some embodiments, the subject receiving treatment has a gram-positive bacterial skin infection, a staphylococcal skin infection, an S. aureus skin infection (Wang et al., Molecules 2018 Nov. 22; 23 (12): 3056; Singh et al., Infect Immun 2023 Jan. 24; 91 (1): e0025222). In some embodiments, the subject receiving treatment has acne. In some embodiments, the subject receiving treatment has advanced glycation end-product accumulation.

In some embodiments, the subject receiving treatment is an adult. In some embodiments, the subject receiving treatment is a child. In some embodiments, the subject has an inflammatory skin condition (e.g., psoriasis) but is otherwise healthy. In some embodiments, the subject has one or more comorbidities.

### V. Kits

Also provided is a kit comprising the formulation as described herein and instructions for use. The kit can be used to perform the methods and uses as described herein, for example, a method of treating a subject in need thereof or for use in the manufacture of a medicament in treating a skin inflammation (e.g., psoriasis). In some embodiments, the subject is a human. The terms subject, individual, and patient are used interchangeably.

### EXAMPLES

### Example 1: Phenylbutyrate Solubility and Exemplary Formulations

In this example, the solubility of sodium phenylbutyrate is characterized.

The solubility of PB in various solvents and excipients was tested and the results are shown in FIG. 8. These data demonstrated that PB (the active pharmaceutical ingredient, API) is highly soluble in hydrophilic solvent and poorly soluble in hydrophobic excipients.

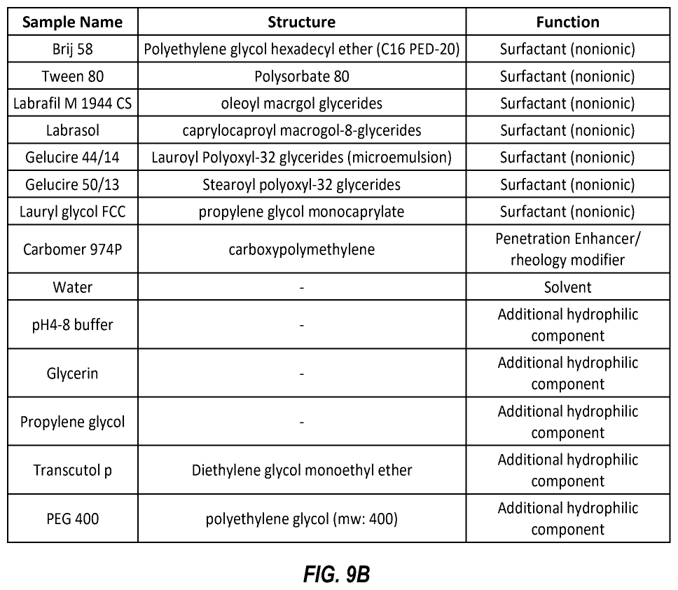

Different ingredients having different phase properties were selected for further investigation based on PB solubility and the physical properties of the excipients. The name and structure of exemplary emollients and emulsifiers are shown in FIG. 9A. The name and structure (if applicable) of exemplary non-ionic surfactants, a penetration enhancer (rheology modifier), a solvent, and additional hydrophilic components are shown in FIG. 9B.

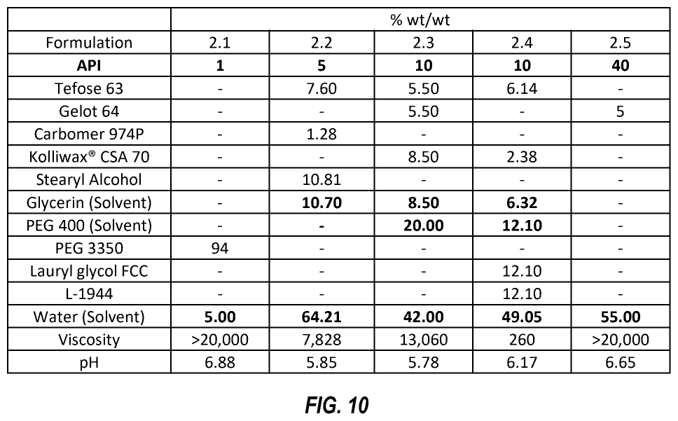

Using two or more of these solvents and/or excipients, the drug loading capacity of PB was tested in five different formulations (i.e., formulations 2.1-2.5) as described in FIG. 10, wherein the amount display refers to the percent weight by weight (% wt/wt). These results suggested that at least 5% hydrophilic component (solvent) is needed to achieve at least 1% drug loading.

### Example 2: Preparation of a Topical Formulation Comprising Phenylbutyrate

In this example, a method of preparing a topical formulation comprising sodium phenylbutyrate (PB) is described, wherein the topical formulation comprising PB is useful in treating patients with psoriasis.

Psoriasis is a chronic inflammatory immune-mediated skin disorder, typically involving distinctive lesions on the scalp and extensor surfaces of the limbs, especially the elbows, knees, and shins. Further, psoriatic skin loses water eight to ten times faster than normal skin. Topical formulations (e.g., such as a topical cream) were considered.

The topical formulation comprised sodium phenylbutyrate, Gelot™ 64 (Mono and Diglycerides and Polyoxyl stearate), Kolliwax® CSA 70 (cetostearyl alcohol), glycerin, 20% sodium thiosulfate solution, sodium benzoate, and water. Of those ingredients, sodium phenylbutyrate, glycerin, sodium thiosulfate solution 20% and sodium benzoate were freely water soluble. Gelot™ 64 and Kolliwax® CSA 70 were solid emulsifiers. Gelot™ 64 had a melting point between 55.5° C. and 62.5° C. and Kolliwax® CSA 70 has a melting point between 49° C. and 56° C. Therefore, the method of preparation was critical for forming a uniform topical formulation (e.g., topical cream).

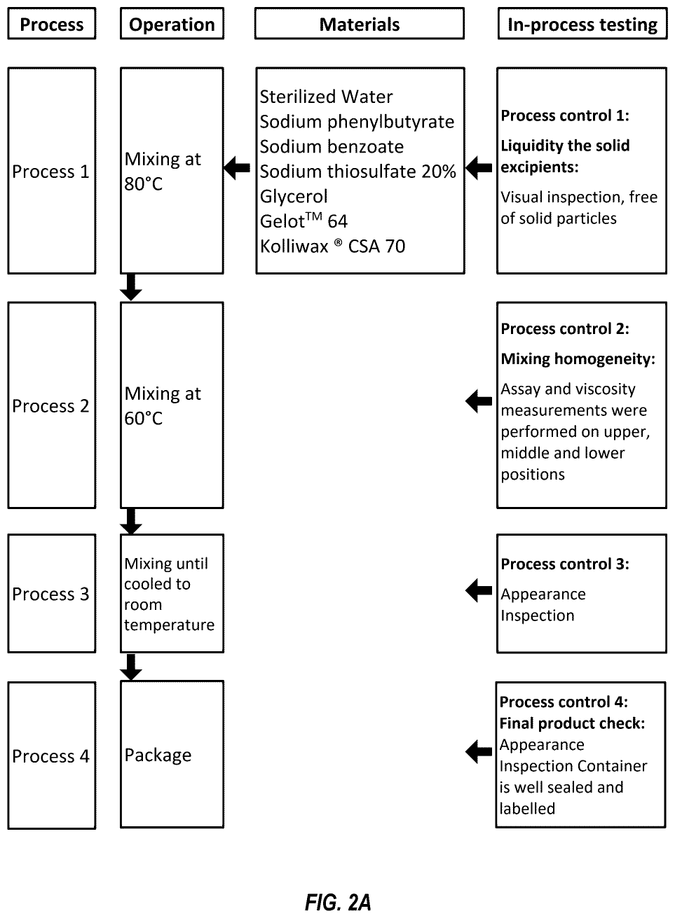

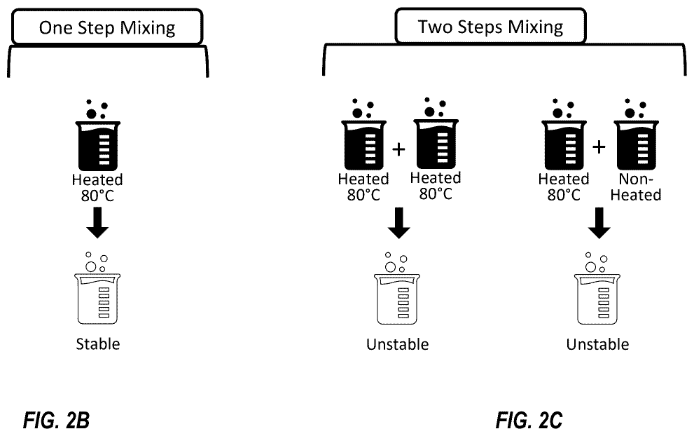

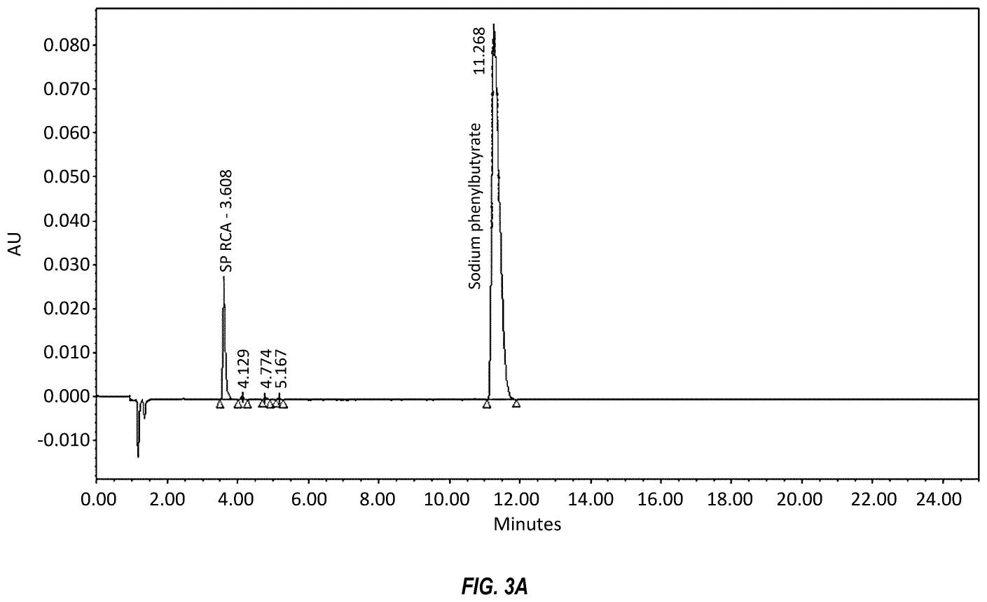

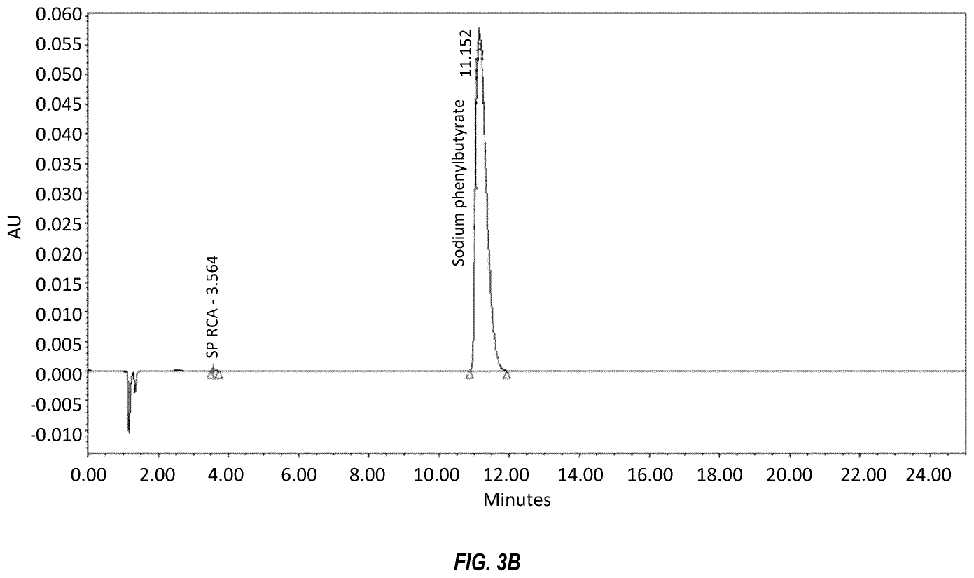

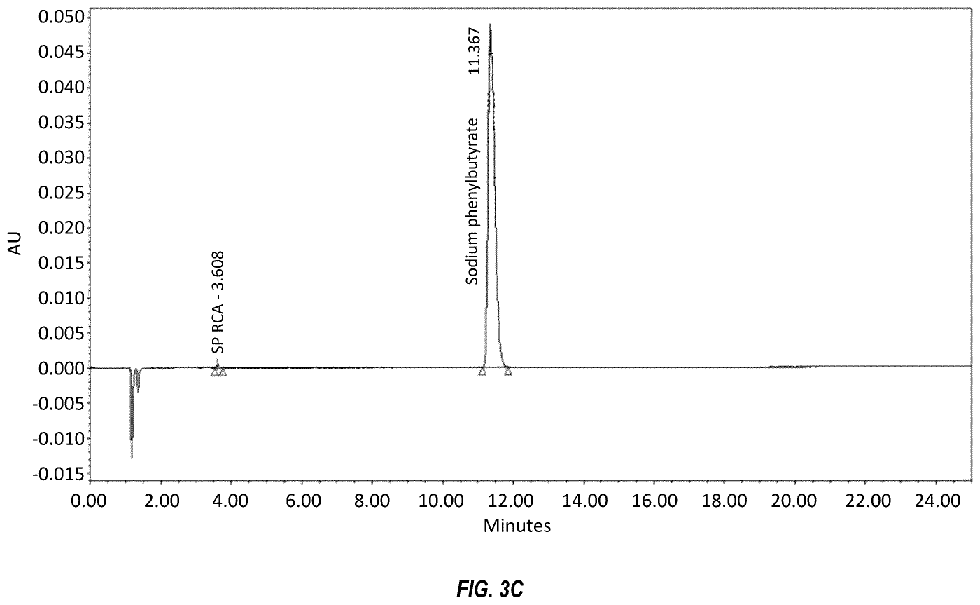

A general workflow of the one-step mixing method for preparing the formulation comprising phenylbutyrate is shown in FIG. 2A. Use of a one-step mixing method (FIG. 2B) produced a stable formulation, while use of a two-step mixing method produced unstable mixtures (FIG. 2C). Using the one-step mixing method, into an empty, clean container, 683 g sterilized water and 100 g sodium phenylbutyrate were added and mixed for 5 minutes at 150 rpm. To this container, 60 g glycerol, 5 g sodium thiosulfate solution 20% and 2 g sodium benzoate were added and mixed for 5 minutes at 150 rpm. To this, 50 g mono and diglycerides, polyoxyl stearate (Gelot™ 64) and 100 g cetostearyl alcohol (Kolliwax® CSA 50) were then added into the same container and then mixed at 400 rpm for 90 minutes at 60° C. An in-process control was performed to ensure the uniformity of the topical formulation (e.g., topical cream). Finally, the topical formulation was formed and allowed to cool to room temperature for 2-3 hours. The peristaltic pump was used in the filling process, by heating the topical formulation (e.g., topical cream) to 60° C. and mixing at 400 rpm, and then filling it into the package material with pumping speed at 30±0.5 rpm.

The resulting topical formulation was validated using tests and specifications that were intended to ensure the identity, purity, and quality of the drug formulation. This included a visual inspection, active pharmaceutical ingredient (API) identification, content uniformity, skin retention assay, impurity analysis, pH and viscosity assessment, in vitro release test, microbial limit analysis, and preservative analysis.

A visual inspection of the topical formulation was conducted wherein the acceptance criteria was white to off white semi-solid.

The presence of the topical formulation API was assessed by ultra-performance liquid chromatographic (UPLC) to confirm that the retention time of the sample solution (i.e., a sample of the topical formulation) was similar to the standard solution containing sodium phenylbutyrate. The UPLC method was an isocratic reversed phase separation. The UPLC method was done using a Waters CORTECS T3 C18, 2.1×150 mm, 2.7 μm, or equivalent column with MeOH:deionized water:Glacial acetic acid=44:55:1 (v/v/v) at 35±5° C. The sample temperature was 10° C. The flow rate was 0.2 mL/min. The detection method was UV at wavelength 260 nm for API identification. The run time was 16 minutes and the injection volume was 8 μL.

The topical formulation was assessed for content uniformity using an ultra-performance liquid chromatographic (UPLC) method, wherein the acceptance criteria was 90.0-110.0% of label claim. The UPLC method was an isocratic reversed phase separation. The UPLC method was done using a Waters CORTECS T3 C18, 2.1×150 mm, 2.7 μm, or equivalent column with MeOH:Deionized water:Glacial acetic acid=44:55:1 (v/v/v) at 35±5° C. The sample temperature was 10° C. The flow rate was 0.2 mL/min. The detection method was UV at wavelength 260 nm for content uniformity. The run time was 16 minutes and the injection volume was 8 μL.

The topical formulation was assessed using an ultra-performance liquid chromatographic (UPLC) method, wherein the acceptance criteria was 90.0-110.0% of label claim. The UPLC method was an isocratic reversed phase separation. The UPLC method was done using a Waters CORTECS T3 C18, 2.1×150 mm, 2.7 μm, or equivalent column with MeOH:Deionized water:Glacial acetic acid=44:55:1 (v/v/v) at 35±5° C. The sample temperature was 10° C. The flow rate was 0.2 mL/min. The detection method was UV absorption at 260 nm. The run time was 16 minutes and the injection volume was 8 μL.

The presence of topical formulation impurities were assessed by ultra-performance liquid chromatographic (UPLC) to confirm that identified and predicted impurities were below a defined threshold. The UPLC method was an isocratic reversed phase separation. The UPLC method was done using a Waters CORTECS T3 C18, 2.1×150 mm, 2.7 μm, or equivalent column with MeOH:Deionized water:Glacial acetic acid=44:55:1 (v/v/v) at 35±5° C. The sample temperature was 10° C. The flow rate was 0.2 mL/min. The detection method was UV absorption at 245 nm for degradation products. The run time was 40 minutes and the injection volume was 4 μL. The topical formulation was assessed to confirm (1) phenylbutyrate related compound A was no more than 0.15% of the total sample by weight, (2) phenylbutyrate related compound B was no more than 0.15% of the total sample by weight, (3) unspecified impurity was no more than 0.10% of the total sample by weight, and (4) total impurities were no more than 0.50% of the total sample by weight.

The pH of the topical formulation was confirmed to be between about 5.0 and about 7.0 using standard procedures. A 125 mg sample of the topical formulation was mixed with 50 mL distilled water before measuring the pH of the resulting mixture.

Rheological properties of the topical formulation were investigated using a cone type rheometer (DV-2T Ultra, Brookfield engineering laboratory, USA) equipped with a circulating system for temperature control. Rheological measurements were made at 25±1° C. using a cone spindle. The topical formulation was confirmed to have a viscosity between about 4000 centipoise (cps) and about 5000 cps.

An in vitro release test (IVRT) and high-performance liquid chromatographic (HPLC) method was used to confirm that not less than 50% release at 6 hours according to IVRT testing conditions (as described in Example 4). About 0.5 g of the samples were placed on a synthetic nylon membrane, and a receptor chamber was filled with a medium. Once the sample was placed on the synthetic nylon membrane, put it on top of the receptor chamber, placed the donor chamber on the membrane, and then clamped the receptor and donor chambers. About 1 mL of the medium samples was taken at pre-determined times (e.g., 1, 2, 3, 4, 5, and 6 hours), and the same amount of the medium was replaced.

A microbial limit test was conducted on the topical formulation, and the microbial limit test confirmed that (1) the Escherichia coli test was negative, (2) the total yeast and mold count (TYMC) was not more than 100 colony forming units (cfu)/mL, (3) the total aerobic microbial count (TAMC) was not more than 1000 cfu/mL, and (4) the Staphylococcus aureus test was negative.

The concentration of preservative in the topical formulation was assessed by HPLC and was confirmed to be not more than 2.2 μg/mg. A Waters Acquity UPLC H-Class system with UV detector was used with a Waters CORTECS T3 C18, 2.1×150 mm, 2.7 μm, or equivalent column with MeOH:Deionized water:Glacial acetic acid=44:55:1 (v/v/v) at 35±5° C. The sample temperature was 10° C. The flow rate was 0.2 mL/min. The detection method was UV absorption at 230 nm. The run time was 16 minutes and the injection volume was 8 μL. The amount of sodium benzoate was calculated according to the following equation:

$$μ g / mg = \frac{{A}_{SAM}}{{A}_{STD}} \times \frac{{D}_{SAM}}{{D}_{STD}} \times \frac{{W}_{STD}}{{W}_{SAM}} \times {P}_{as is} \times 1000$$

ASAM=The peak area of sodium benzoate in the sample solution
ASTD=The average peak area of sodium benzoate obtained from the standard working solution
WSAM=Actual sample weight (mg) of sample preparation
WSTD=The weight (mg) of sodium benzoate standard stock solution
DSAM=The dilution factor of the sample solution
DSTD=The dilution factor of standard solution
Pas is =Purity Factor of sodium benzoate reference standard

### Example 3: In Vitro Skin Permeation and Retention Testing

The skin permeation and retention studies were performed by using human cadaver skins as an in vitro mimic for the pharmacokinetic behavior of the drug product in vivo (FIG. 4). Cadaver skins were mounted on a modified Franz cell systems equipped with a water bath for maintaining the constant temperature at 37° C. A magnetic stir bar was placed in the receptor chamber to stir constantly to ensure solution uniformity. The human cadaver skin was stored in a−80° C. freezer, and the cadaver skin was thawed and washed in purified water prior to use. Full thickness skin was cut into a round shape with a radius of 1.6 cm. The skin was then mounted on the Franz cell. Once the pre-cut skin was placed on the donor chamber, the donor chamber and the receptor chamber were clamped together. Phosphate buffer pH 6.8 was used to fill to the marked line (14 mL) with a needled syringe. The formulations to be tested were then applied onto the skin on the donor chamber. A piece of release liner was placed on the formulation to ensure the topical formulation was dispersed evenly on the skin. At 1-, 2-, 4-, 6-, and 24-hour timepoints, 1 mL test samples were withdrawn and analyzed by the HPLC for the concentration of sodium phenylbutyrate.

$$Cumulative Amount Permeated = Vs ( {\sum }_{n = 1}^{n} Cn - 1 ) + Cn \times Vr \qquad ( Equation 1 )$$

C1, C2 . . . Cn=sample concentrations in samples 1, 2, . . . n,
Vr=volume of the receiver solution
Vs=the volume of the sample

$$Flux = \frac{( Cumulative Amount Permeated at time {T}_{n + 1} - Cumulative Amount Permeated at time {T}_{n} )}{( {T}_{n + s} - {T}_{n} )} \qquad ( Equation 2 )$$

An extraction method was developed to extract sodium phenylbutyrate from the skin using 100% methanol solution. Once the in vitro permeation and retention studies were completed, a mass balance study was performed. The topical formulation on the release liner was placed in a glass bottle, and 100% methanol solution was added to extract the sodium phenylbutyrate from the release liner. The topical formulation remaining on the surface of the skin was wiped with a kimwipe, and the kimwipe was placed in a glass bottle. To this, 100% methanol solution was added to extract the sodium phenylbutyrate from the kimwipe. Lastly, the skin was placed in a glass bottle and then 100% methanol was added to extract the sodium phenylbutyrate. The collected samples were analyzed by the HPLC for the concentration of sodium phenylbutyrate according to the HPLC conditions described below:

HPLC conditions:(a) Instrument-Waters e2695 HPLC system with UV detector; (b) Column-InertSustain C8, 5 μm, 4.6× 50 mm, or equivalent; (c) Mobile phase: MeOH:Deionized water:Glacial acetic acid=49:50:1 (v/v/v); (d) Column Temperature: 40±5° C.; (e) Sample Temperature: 10° C.; (f) Flow Rate: 1.2 mL/min; (g) Detection: UV absorption: 260 nm; (h) Run Time: 5 minutes; and (i) Injection Volume: 20 μL.

The main peak of the sodium phenylbutyrate reference standard was required to elute with a retention time of 2.8±10% minutes.

The amount of sodium phenylbutyrate on the surface of the skin (skin retention, equation 4) was the total amount of sodium phenylbutyrate extracted from the release liner and from the kimwipe. The amount of sodium phenylbutyrate retained inside the skin (on the skin, equation 5) was the amount of sodium phenylbutyrate extracted from the skin. The amount of sodium phenylbutyrate permeated through the skin (permeated, equation 6) was the total amount of sodium phenylbutyrate from the permeation samples.

The mass balance equation was summarized below:

$$Skin retention + On the Skin + Permeated = Total amount of sodium phenylbutyrate \qquad ( Equation 3 )$$

$$\% Skin retention = \frac{Skin retention}{Total amount of sodium phenylbutyrate} \qquad ( Equation 4 )$$

$$\% On the Skin = \frac{On the Skin}{Total amount of sodium phenylbutyrate} \qquad ( Equation 5 )$$

$$\% Permeated = \frac{Permeated}{Total amount of sodium phenylbutyrate} \qquad ( Equation 6 )$$

“% Skin retention”=the amount of sodium phenylbutyrate extracted from the skin which was the retained amount inside the skin

“% On the Skin”=amount of sodium phenylbutyrate on the surface of the skin which was extracted from the release liner and the kimwipe.

“% Permeated”=amount of sodium phenylbutyrate permeated through the skin which was the total amount of sodium phenylbutyrate from the permeation samples.

### Results

As shown in Table E1, Formulation 1 achieved the highest local concentration of phenylbutyrate (PB) (2.35 mg). Formulation 3 showed the second highest local concentration of PB (2.35 mg), but also significantly elevated levels of systemic PB (2.33 g) compared to the other two formulations (0.54 mg and 0.40 mg, respectively). Based on these data, it was determined that the local skin tissue phenylbutyrate concentration for formulations 1, 2, and 3 was 13.72, 8.17, and 8.99 mmole/1000 cm³, respectively. As described in Example 5, these formulations were stable under stressed storage conditions (e.g., 60° C. for 21 days).

TABLE E1
Skin retention and permeation assay results
using formulations 1, 2, and 3

| Formulation No. | On Skin (mg) | In Skin (mg) | Systemic (mg) |
|---|---|---|---|
| 1 | 12.76 | 2.35 | 0.54 |
| 2 | 15.81 | 1.40 | 0.40 |
| 3 | 14.49 | 1.54 | 2.33 |
| Control | 6.87 | 0.95 | 0.00 |

These data demonstrate that topical formulations comprising phenylbutyrate can achieve high local tissue concentrations with limited systematic exposure.

### Example 4: In Vitro Release Test (IVRT) for Topical Formulation

In this example, an in vitro release test (IVRT) and high-performance liquid chromatographic (HPLC) method were developed to determine the release of sodium phenylbutyrate from a topical formulation comprising sodium phenylbutyrate.

An in vitro release test (IVRT) has the potential to establish the differences in the drug release rates among the formulations with the altered compositions. This method was created to serve as a simple and reliable aid during product development and also as a quality control tool in assessing stability, aging, and batch-to-batch uniformity of the topical formulation. In accordance with FDA, during a 4-6-hour assay period, sampling at least hourly, the linearity of cumulative amount released plotted versus the square root of time (Vt) should not be less than 0.95. In addition to the FDA guidance, and in accordance with EMA guidance, at least 70% release of the active substance should be achieved at the last time point.

The IVRT was performed by Franz Vertical Diffusion Cell apparatus with 14 mL of medium (75% MeOH:THF=87:13 (v/v)), as shown in FIG. 5A, and the collected samples were assayed by a HPLC method using an isocratic reversed phase separation (using a Waters e2695 HPLC system with UV detector with an InertSustain C8, 4.6×50 mm, 5 μm, or equivalent column) and UV absorption at 260 nm. A topical formulation sample of about 0.5 g was placed on a nylon membrane (0.45 μm). The receptor chamber was filled with about 12 mL of medium and a stir bar. Once the sample was placed on a nylon membrane and then warmed to 32±1° C., the nylon membrane with sample was placed on top of the receptor chamber. To this, the donor component was placed on top of the receptor chamber, and the receptor and donor components were clamped together to start the assay. At 1-, 2-, 3-, 4-, 5-, and 6-hour timepoints, about 1 mL of the medium sample was collected for HPLC analysis of sodium phenylbutyrate. The same amount of the medium was then replaced in the receptor chamber.

HPLC conditions:(a) Instrument-Waters e2695 HPLC system with UV detector; (b) Column-InertSustain C8, 5 μm, 4.6×50 mm, or equivalent; (c) Mobile phase: MeOH:Deionized water:Glacial acetic acid=49:50:1 (v/v/v); (d) Column Temperature: 40±5° C.; (e) Sample Temperature: 10° C.; (f) Flow Rate: 1.2 mL/min; (g) Detection: UV absorption: 260 nm; (h) Run Time: 5 minutes; and (i) Injection Volume: 20 μL.

The main peak of the sodium phenylbutyrate reference standard was required to elute with a retention time of 2.8±10% minutes.

### Results

Three candidate formulations were examined with the IVRT method to determine and compare percent drug release profiles. The composition of each formulation is shown in FIG. 5B, the R2 correlation between average release and sampling time was for each formulation was above the predetermined threshold, wherein formulation 1, 2, and 3 had an R2 correlation between average release and square root of sampling time of 0.978, 0.9742, and 0.9874, respectively. For each formulation, the percentage drug release was asymptotic across the 6 sampling time points, where the max percentage drug release achieved formulation 1, 2, and 3 was about 64%, about 55%, and about 65%, respectively (FIG. 5C).

These data demonstrate that the topical formulation comprising PB evaluated in this example, and described in Example 2, can be effectively delivered to its target in an amount expected to have efficacy in patients with skin inflammatory disorders such as psoriasis.

### Example 5: Formulation Stability Testing

In this example, an accelerated stability study and a long-term stability study were conducted for the topical formulations comprising sodium phenylbutyrate (PB), as described in Example 2. The stability evaluation included an evaluation of the physical and chemical characteristics of the active drug substance, covering the expected duration of the shelf life and retest period. These studies were designed to increase the rate of chemical degradation or physical change of the active pharmaceutical ingredient (API) phenylbutyrate by using exaggerated storage conditions as part of the formal, definitive, stability protocol.

For the long-term stability studies storage, all samples were stored in an upright position at 25±2° C. and 60±5% relative humidity (RH). The date the sample was placed in the described storage conditions was considered as Time Zero (To) for the long-term stability study. Samples were collected over time, including at 1, 3, 6, and 12 months, and the appearance, identification, impurity, in vitro release studies, viscosity, preservative, and physical properties were examined for each sample.

For the accelerated stability studies storage, all samples were stored in an upright position at 40±2° C. and 75±5% relative humidity (RH). The date the sample was placed in the described storage conditions was considered as Time Zero (To) for the accelerated stability study. Samples were collected over time, including at 1, 3, and 6 months, and the appearance, identification, impurity, in vitro release studies, viscosity, preservative, and physical properties were examined for each sample.

Significant changes for the stability sample evaluation were defined as (1) a 5% potency loss from the initial assay value of a batch, (2) any specified degradant exceeding the predetermined specification limit, and (3) appearance and physical properties not matching the acceptance criteria.

### Results

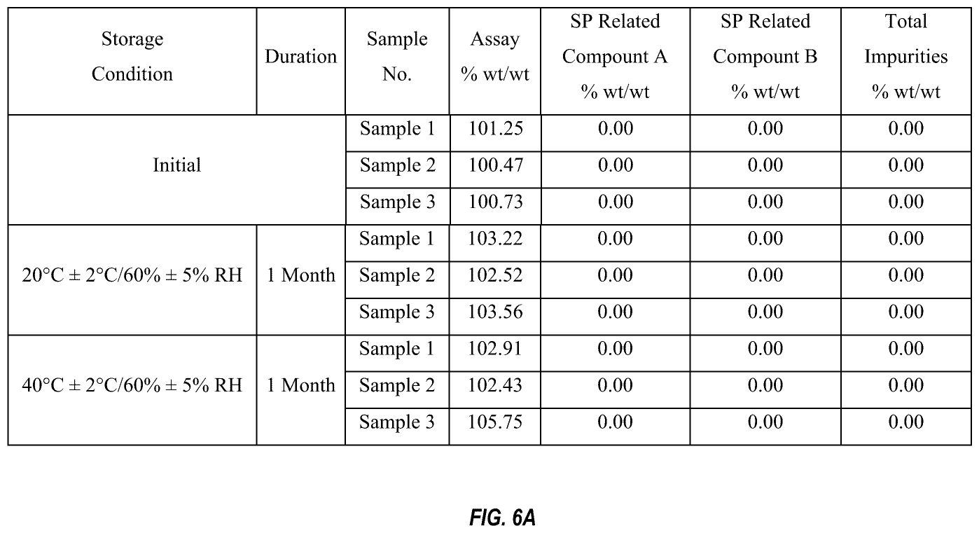

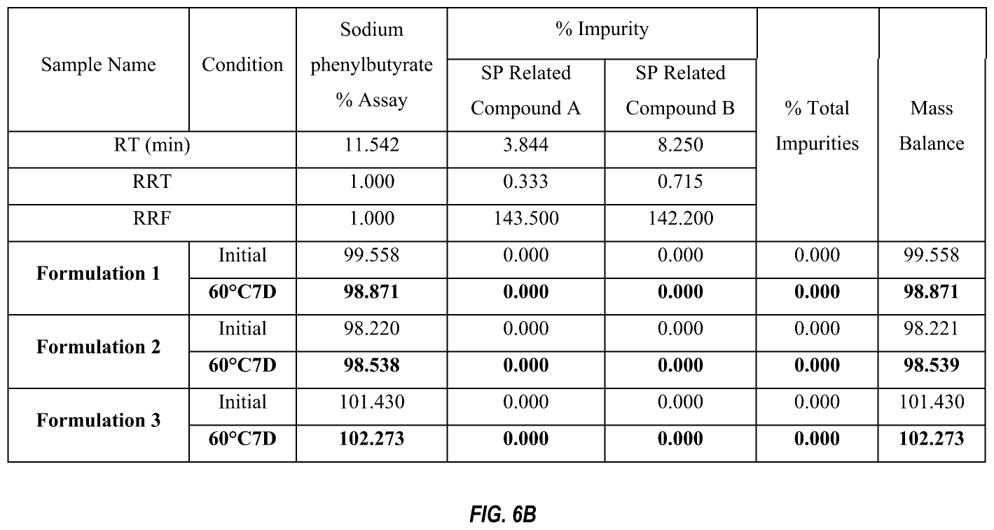

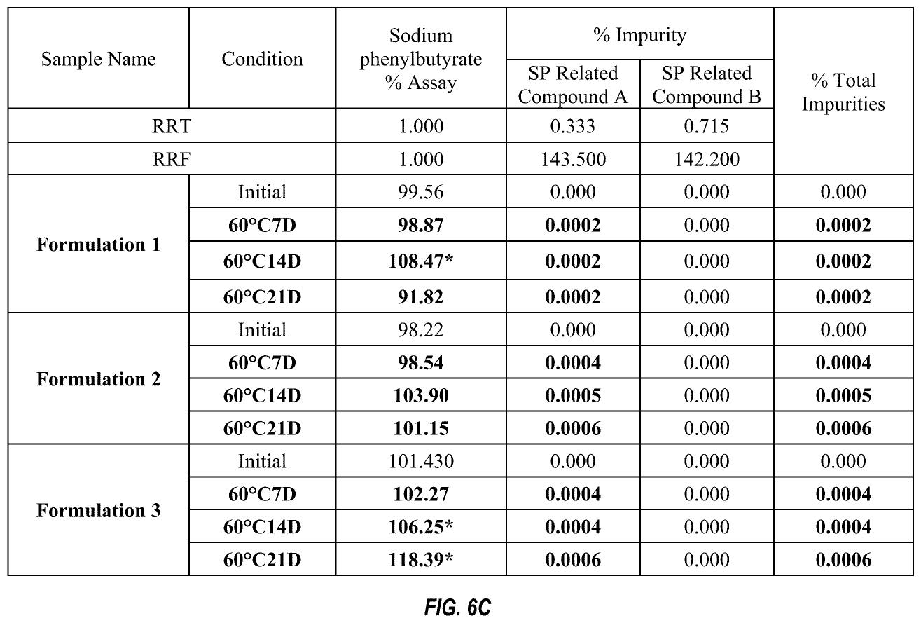

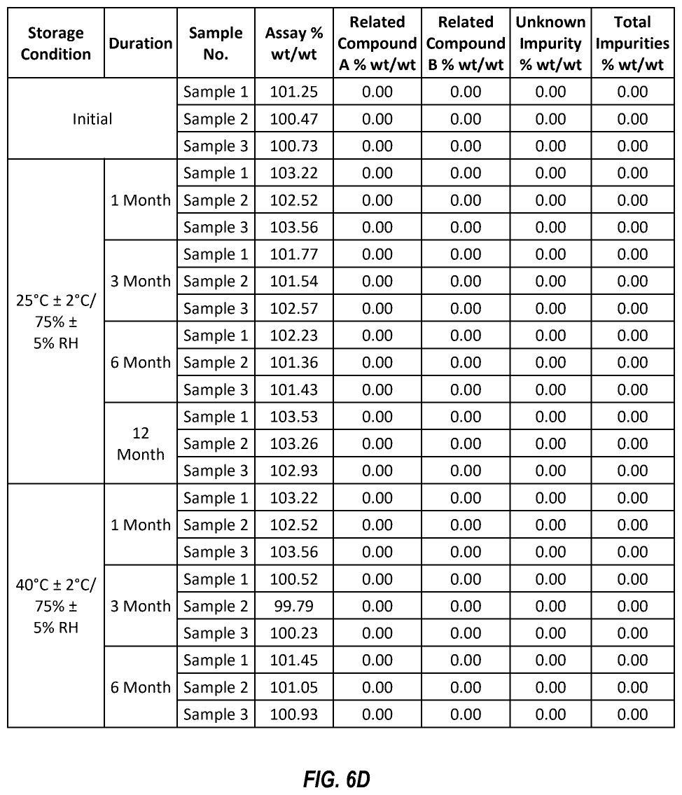

Samples of formulation 1 were collected after 1 month of storage under (1) 25±2° C. and 60±5% relative humidity (RH) or (2) 40±2° C. and 75±5% RH. Samples were analyzed by HPLC for phenylbutyrate (PB) and related impurities. As shown in FIG. 6A, the level of PB is consistent with the input and control amounts, while specific impurities (e.g., related compound A) and total impurities after stress storage were 0.00 in every instance across the three biological replicate samples.

In another accelerated stability study, formulation 1 samples were collected after 1, 3, 6, and 12 months of storage under (1) 25±2° C. and 60±5% relative humidity (RH) or after 1, 3, and 6 months of storage under (2) 40±2° C. and 75±5% RH. Samples were analyzed by HPLC for phenylbutyrate (PB) and related impurities. As shown in FIG. 6D, the level of PB is consistent with the input and control amounts, while specific impurities (e.g., related compound A) and total impurities after stress storage were 0.00 in every instance across the three biological replicate samples.

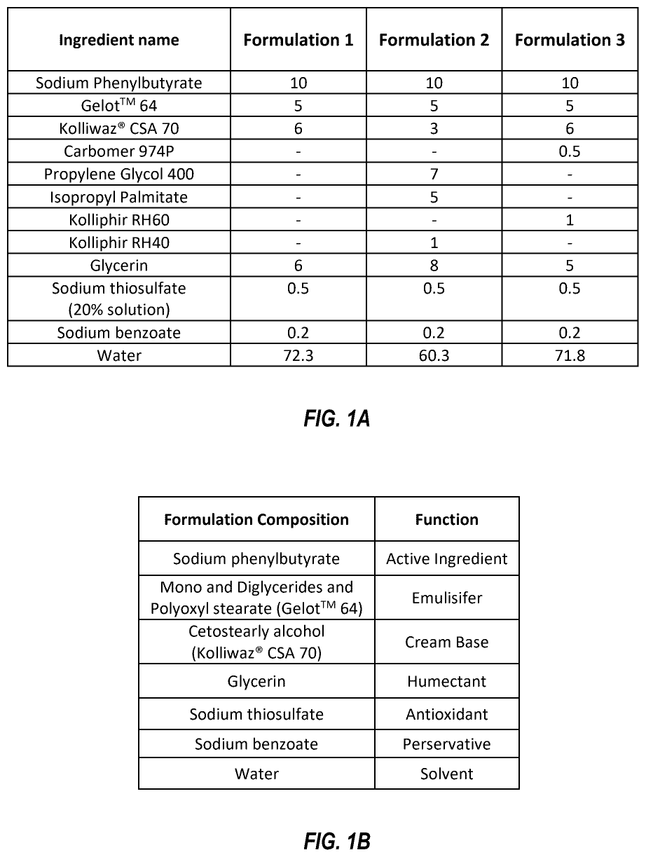

In a separate 7-day accelerated stability study, formulation 1, 2, and 3 (FIG. 1) were stored under extreme stress condition (60° C.) for 7 days. As shown in FIG. 6B, the level of PB, total impurities, and mass balance after stress storage were acceptable in all three formulations. All three formulations also passed centrifuge and freeze/thaw challenges as well (data not shown).

In another separate 21-day accelerated stability study, formulation 1, 2, and 3 (FIG. 1) were stored under stress condition (60° C.) for 21 days using small scale samples (20 g). As shown in FIG. 6C, the level of PB and total impurities after stress storage were acceptable in all three formulations.

These data demonstrate that the topical formulations comprising phenylbutyrate (PB) are stable under mild and extreme stress storage conditions, suggesting that the topical formulations described herein may have extended shelf lives without decline in produce safety or efficacy. These data can be used to assess longer chemical effects at non-accelerated conditions and to evaluate the impact of short-term excursions outside the label storage conditions such as might occur during shipping.

### Example 6: Validation of Formulations with Minor Modifications

In this example, pharmaceutical formulations comprising sodium phenylbutyrate (PB) were optimized for larger scale batch production (e.g., formulations 1.1 and 1.2), wherein the pharmaceutical formulations were validated for physical, chemical, and function integrity compared to formulation 1.

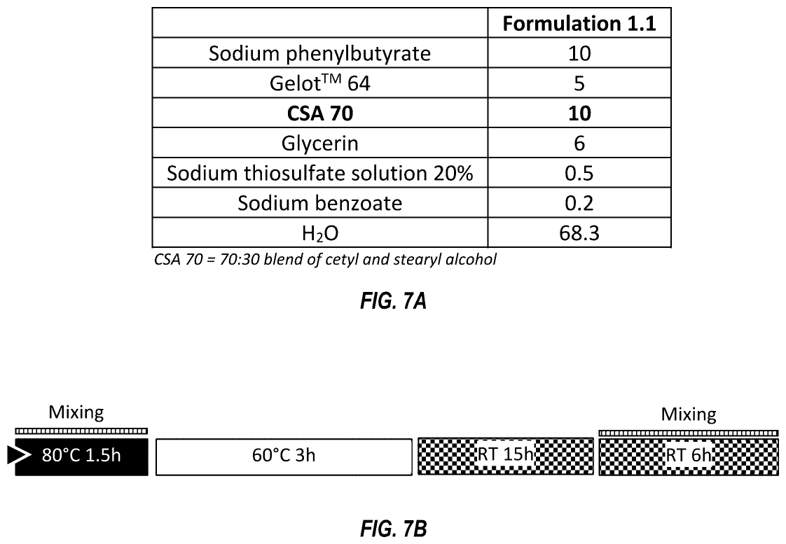

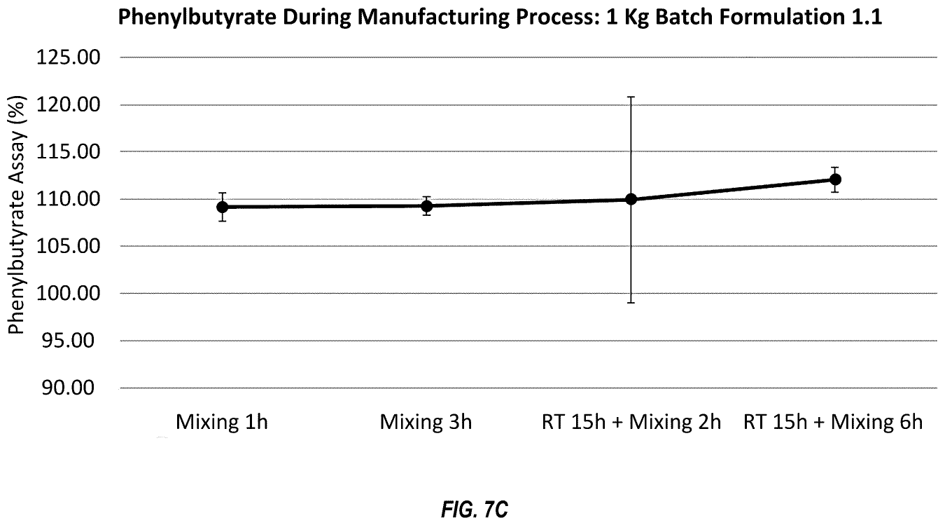

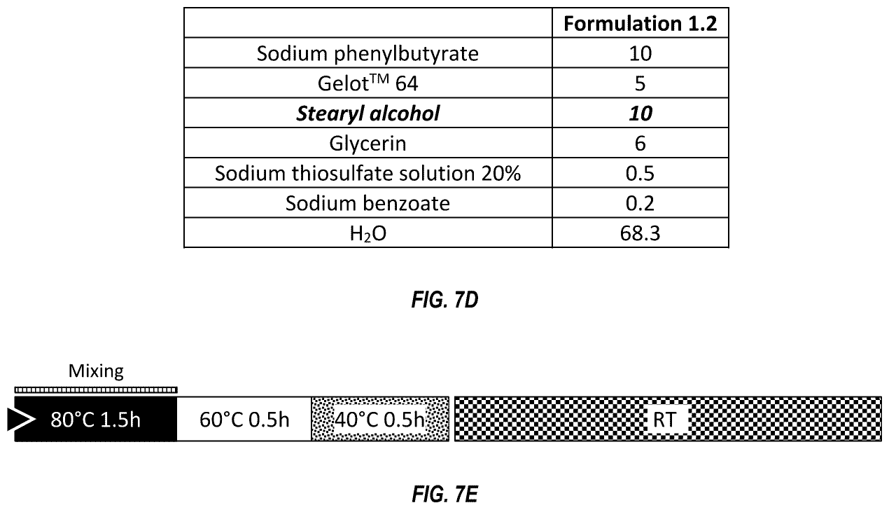

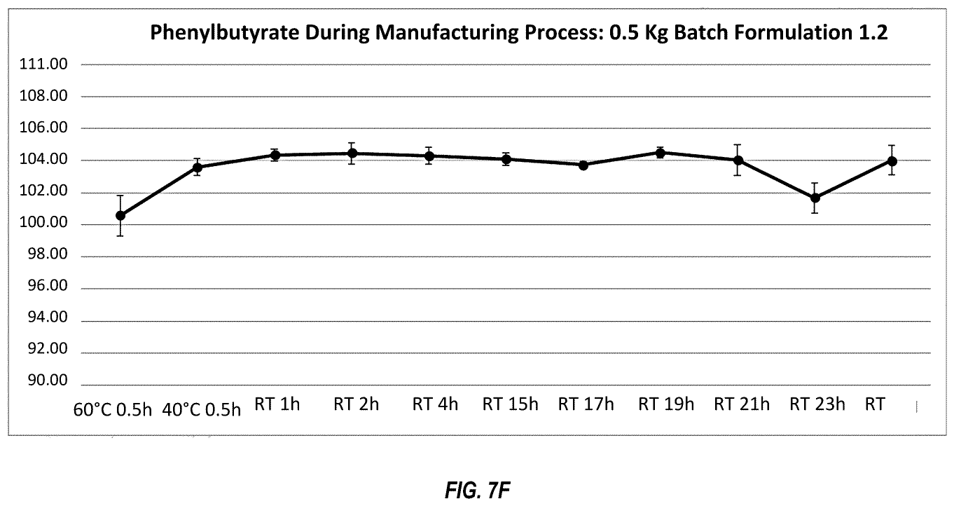

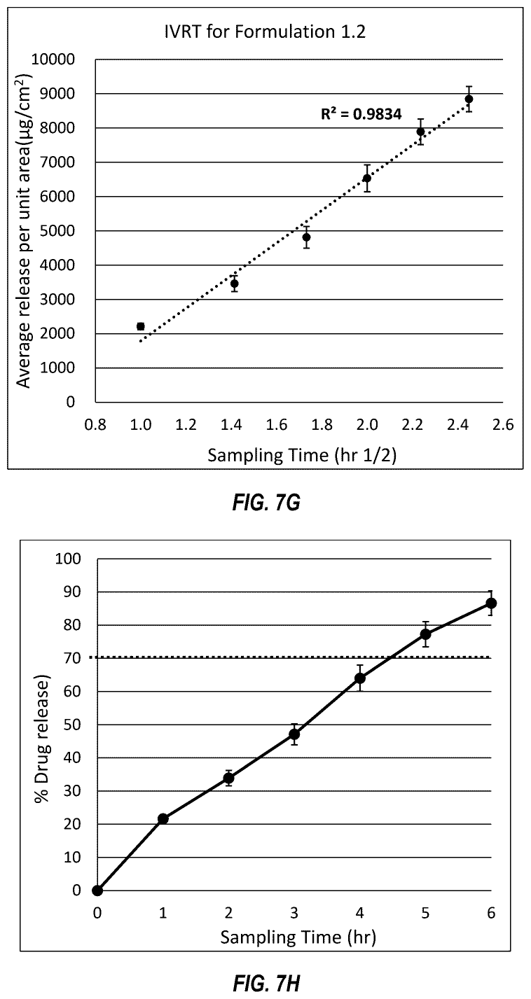

Formulation 1.1, comprising the ingredients listed in FIG. 7A, was prepared generally according to the methods described in Example 2. The methods used for preparation of formulation 1.1 differed from the methods presented in Example 2 as shown in FIG. 7B. Briefly, the one-pot mixture was mixed and heated for 90 minutes at 80° C. The mixture was further heated for 3 hours at 60° C., and then allowed to cool at room temperature for 15 hours prior to mixing for 6 hours at room temperature. At the conclusion of this final mixing period, the mixture was filed into packaging material as described in Example 2.

During the manufacturing process, formulation 1.1 was assessed for phenylbutyrate levels compared to control (e.g., formulation 1) (FIG. 7C). Phenylbutyrate levels were consistent across the manufacturing process, suggesting that the minor modifications of formulation 1.1 do not impact the integrity of phenylbutyrate.

Next, formulation 1.2, comprising the ingredients listed in FIG. 7D, was prepared generally according to the methods described in Example 2. The methods used for preparation of formulation 1.1 differed from the methods presented in Example 2 as shown in FIG. 7E. Briefly, the one-pot mixture was mixed and heated for 90 minutes at 80° C. The mixture was further heated for 0.5 hours at 60° C. and then lightly heated for 0.5 hours at 40° C., before an extended cooling period at room temperature. At the conclusion of this cooling period, the mixture was filed into packaging material as described in Example 2.

During the manufacturing process, formulation 1.2 was assessed for phenylbutyrate levels compared to control (e.g., formulation 1) (FIG. 7F). Phenylbutyrate levels were consistent across the manufacturing process, suggesting that the minor modifications of formulation 1.2 do not impact the integrity of phenylbutyrate.

Formulation 1.2 was validated for physical, chemical, and function integrity compared to formulation 1 and internal thresholds, as described in the previous examples. A summary of the available validation results are described in Table E2. These results validated formulation 1.2 as being very similar to formulation 1 as characterized in the previous examples.

TABLE E2
Formulation 1.2 test and validation summary

| Attribute | Acceptance Criteria | Result | |
|---|---|---|---|
| Appearance | White to off-white semisolid | Conforms | |
| Identification | Sodium phenylbutyrate: the | Conforms | |
| | retention time of the sample | | |
| | solution is similar | | |
| | to the standard solution. | | |
| Content | 90.0-110.0% of label claim | 99.78% | |
| Uniformity | | | |
| Phenylbutyrate | 90.0-110.0% of label claim | 100.81% | |
| Impurity | 1. Phenylbutyrate related | 1. | ND. |
| | compound A: ≤0.15% | | |
| | 2. Phenylbutyrate related | 2. | ND |
| | compound B: ≤0.15% | | |
| | 3. Unspecified impurity: ≤0.10% | 3. | ND |
| | 4. Total impurities, ≤0.50% | 4. | 0.00% |
| IVRT | ≥50% at 6 hrs | 86.63% | |
| Preservative | ≤2.2 μg/mg | 1.96 | μg/mg |
| pH | 5.0~7.0 | 6.828 | |
| Viscosity | 4500 cps~5500 cps | 4793 | cps |
| Microbial limit | 1. Escherichia coli, Negative | 1. | ND. |
| test | 2. TYMC, ≤100 cfu/mL | 2. | ND |
| | 3. TAMC, ≤1000 cfu/mL | 3. | ND |
| | 4. Staphylococcus aureus, Negative | 4. | ND |

Results from the formulation 1.2 in vitro release test (IVRT) analysis reported in Table E2 are shown in FIGS. 7G-7H (methods as described generally in Example 4). Timepoint samples collected during the 6-hour analysis period showed excellent drug release results. As shown in FIG. 7G the R2 correlation between average release and square root of sampling time was 0.9834. The percentage drug release was linear across the 6 sampling time points, where between the 4- and 5-hour timepoints, the percentage drug release achieved 70% (FIG. 7H). At the 5-hour timepoint, the percentage drug release was about 78% and at the 6-hour timepoint, the percentage drug release was about 86.63% (FIG. 7H).

Additionally, an in vitro skin retention assay was performed using formulation 1.2, according to the methods described in Example 3. The results from this in vitro skin retention assay are shown in Table E3. Samples from a 3 kg batch and a 0.6 kg batch of formulation 1.2 were examined. The 3 kg formulation batch and the 0.6 kg formulation batch both achieved high local concentration of sodium phenylbutyrate (2.79 mg and 2.45 mg, respectively). Both formulation batches had elevated levels of systemic sodium phenylbutyrate as well (more than 2 mg). Overall, these data are consistent with skin permeation results obtained for smaller batch sizes.

TABLE E3
Skin retention and permeation assay results using formulation 1.2

| Formulation No. | On Skin (mg) | In Skin (mg) | Systemic (mg) |
|---|---|---|---|
| 1.2 (3 kg) | 12.15 | 2.79 | 2.03 |
| 1.2 (0.6 kg) | 11.07 | 2.45 | 2.26 |
| Control | 6.41 | 0.51 | 0.21 |

These data demonstrate that pharmaceutical formulations with minor modifications for larger batch manufacturing are physically, chemically, and functionally comparable to formulation 1. Formulations 1.1 and 1.2 demonstrated consistent phenylbutyrate levels during manufacturing, and multiple larger batch samples of formulation 1.2 were further validated.

1. A pharmaceutical formulation comprising an optionally 4-substituted phenylbutyrate in an emulsion, wherein:
(a) the phenylbutyrate is present at a concentration ranging from about 1-40% by weight,
the emulsion comprises a hydrophobic phase dispersed in an aqueous phase, and
the formulation is a topical formulation;
(b) the phenylbutyrate is present at a concentration ranging from about 2-40% by weight,
the emulsion comprises a hydrophobic phase and an aqueous phase, and
the formulation is a topical formulation; or
(c) the emulsion comprises a hydrophobic phase and an aqueous phase,
the aqueous phase comprises water and one or more additional hydrophilic components,
the one or more additional hydrophilic components collectively are present in a ratio of 1:1 or greater by weight relative to the optionally 4-substituted phenylbutyrate, and the formulation is a topical formulation.

2. (canceled)

3. (canceled)

4. A pharmaceutical formulation comprising an optionally 4-substituted phenylbutyrate in an emulsion, wherein:
(a) the emulsion comprises a hydrophobic phase and an aqueous phase,
the aqueous phase comprises water and one or more additional hydrophilic components,
the formulation has the property of at least about 40% phenylbutyrate release at 6 hours under in vitro release testing conditions,
and the formulation is a topical formulation;
(b) the emulsion comprises a hydrophobic phase and an aqueous phase,
the aqueous phase comprises water and one or more additional hydrophilic components,
the formulation has the property of at least about 5% phenylbutyrate skin retention at 6 hours under in vitro skin retention assay conditions,
and the formulation is a topical formulation;
(c) the emulsion comprises a hydrophobic phase and an aqueous phase,
the aqueous phase comprises water and one or more additional hydrophilic components,
the formulation has the property of no more than about 12% phenylbutyrate skin permeation at 6 hours under in vitro skin retention assay conditions,
and the formulation is a topical formulation; or
(d) the emulsion comprises a hydrophobic phase and an aqueous phase,
the aqueous phase comprises water and one or more additional hydrophilic components,
the formulation has the property of at least about 1:1 ratio of phenylbutyrate skin retention to skin permeation at 6 hours under in vitro skin retention assay conditions,
and the formulation is a topical formulation.

5.-8. (canceled)

9. The pharmaceutical formulation of claim 1, wherein the hydrophobic phase is dispersed in the aqueous phase.

10. (canceled)

11. (canceled)

12. The pharmaceutical formulation of claim 2, wherein the one or more additional hydrophilic components is chosen from glycerin, polypropylene glycol, propylene glycol, diethylene glycol monoethyl ether, polyethylene glycol and combinations thereof.

13. (canceled)

14. The pharmaceutical formulation of claim 1, wherein the optionally 4-substituted phenylbutyrate is a pharmaceutically acceptable salt, optionally wherein the optionally 4-substituted phenylbutyrate is a sodium salt or a potassium salt.

15. (canceled)

16. (canceled)

17. The pharmaceutical formulation of claim 1, wherein the optionally 4-substituted phenylbutyrate is a 4-halo phenylbutyrate, optionally wherein the optionally 4-substituted phenylbutyrate is 4-chlorophenylbutyrate or 4-iodophenylbutyrate.

18. (canceled)

19. (canceled)

20. The pharmaceutical formulation of claim 1, wherein the phenylbutyrate is unsubstituted, optionally wherein the phenylbutyrate is sodium phenylbutyrate.

21.-24. (canceled)

25. The pharmaceutical formulation of claim 1, wherein the formulation comprises water at a concentration ranging from about 5-80% by weight.

26.-30. (canceled)

31. The pharmaceutical formulation of claim 1, wherein the formulation further comprises a nonionic emulsifier, optionally wherein the nonionic emulsifier comprises glycerol monostearate and/or a PEG stearate, further optionally wherein the nonionic emulsifier is present in the formulation at a concentration ranging from about 1-10% by weight, further optionally wherein the nonionic emulsifier is a combination of glycerol monostearate and a PEG stearate, further optionally wherein the PEG stearate has a molecular weight of about 3-4 kDa, or about 3.5 kDa.

32.-37. (canceled)

38. The pharmaceutical formulation of claim 1, wherein the formulation further comprises an emollient, optionally wherein the emollient is present in the formulation at a concentration ranging from about 1-10% by weight, further optionally wherein the emollient comprises cetyl alcohol and/or stearyl alcohol.

39.-44. (canceled)

45. The pharmaceutical formulation of claim 1, wherein the formulation further comprises an antioxidant, optionally wherein the antioxidant comprises a thiosulfate, further optionally wherein the antioxidant is present in the formulation at a concentration ranging from about 0.1-1% by weight.

46.-50. (canceled)

51. The pharmaceutical formulation of claim 1, wherein the formulation further comprises a preservative, optionally wherein the preservative is present in the formulation at a concentration ranging from about 0.1-1% by weight, optionally wherein the preservative comprises a benzoate, further optionally wherein the preservative is sodium benzoate.

52.-56. (canceled)

57. The pharmaceutical formulation of claim 1, wherein the formulation further comprises at least one additional excipient, optionally wherein the at least one additional excipient comprises a PEG, optionally wherein the PEG is PEG400; a glycerol polyethylene glycol hydroxy stearate; a hydrogenated ethoxylated castor oil, optionally wherein the hydrogenated ethoxylated castor oil is PEG-60 hydrogenated castor oil; and/or glycerin, and further optionally wherein the at least one additional excipient is present in the formulation at a concentration ranging from about 6-8% by weight.

58. (canceled)

59. (canceled)

60. The pharmaceutical formulation of claim 1, wherein the topical formulation is a cream, ointment, gel, spray, or foam, or wherein the topical formulation is contained in a patch or dressing, optionally wherein the patch is an adhesive patch or the dressing is a non-adhesive dressing, an occlusive dressing, or a non-occlusive dressing.

61. (canceled)

62. The pharmaceutical formulation of claim 1, wherein:
(a) the formulation comprises less than about 0.01% by weight of 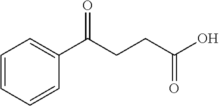 [SMILES: O=C(O)CCC(=O)c1ccccc1]
(related compound A) and salts thereof; and/or
(b) the formulation undergoes less than about 0.01% conversion by weight of the phenylbutyrate to 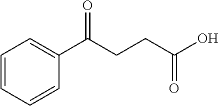 [SMILES: O=C(O)CCC(=O)c1ccccc1]
(related compound A) and salts thereof upon storage at 60° C. for 21 days.

63. (canceled)

64. The pharmaceutical formulation of claim 4, wherein:
(a) the formulation has the property of at least about 50% phenylbutyrate release at 6 hours under in vitro release testing conditions;
(b) the formulation has the property of at least about 40% phenylbutyrate release at 5 hours under in vitro release testing conditions;
(c) the formulation has the property of at least about 40% phenylbutyrate release at 4 hours under in vitro release testing conditions;
(d) the formulation has the property of at least about 40% phenylbutyrate release at 3 hours under in vitro release testing conditions; and/or
(e) the formulation has the property of at least about 40% phenylbutyrate release at 2 hours under in vitro release testing conditions.

65.-69. (canceled)

70. The pharmaceutical formulation of claim 4, wherein the formulation has the property of at least about 10% phenylbutyrate skin retention at 6 hours under in vitro skin retention assay conditions.

71.-73. (canceled)

74. The pharmaceutical formulation of claim 4, wherein the formulation has the property of no more than about 10% phenylbutyrate skin permeation at 6 hours under in vitro skin retention assay conditions.

75. (canceled)

76. The pharmaceutical formulation of claim 4, wherein the formulation has the property of at least about 2:1 ratio of phenylbutyrate skin retention to skin permeation at 6 hours under in vitro skin retention assay conditions.

77. (canceled)

78. The pharmaceutical formulation of claim 1, wherein the formulation has a pH in the range of about 4-8.

79. (canceled)

80. The pharmaceutical formulation of claim 1, wherein the formulation has a viscosity in the range of about 3500-6500 cps.

81. (canceled)

82. The pharmaceutical formulation of claim 1, wherein:
(a) the formulation is stable under stress storage conditions comprising a temperature of at least 25±2° C. and a relative humidity (RH) at least 60±5% for at least 3 months;
(b) the formulation is stable under stress storage conditions comprising a temperature of at least 40±2° C. and a relative humidity (RH) at least 75±5% for at least 3 months;
(c) the formulation is stable under stress storage conditions comprising a temperature of at least 60° C. for at least 7 days; and/or
(d) the formulation is stable under stress storage conditions comprising a temperature of at least 60° C. for at least 21 days.

83.-87. (canceled)

88. A method of treating a skin condition, comprising applying the pharmaceutical formulation of claim 1 topically to a subject having the skin condition.

89.-97. (canceled)

98. The method of claim 88, wherein the skin condition is selected from skin inflammation, an inflammatory skin disorder, psoriasis, epidermolysis bullosa, epidermolysis bullosa simplex, atopic dermatitis, eczema, seborrheic dermatitis, rosacea, toxic epidermal necrolysis, Stevens Johnson syndrome, Lyell syndrome, erythema multiforme, Necrobiosis lipoidica, Peeling skin syndrome, Ichthyosis, Neurodermatitis, Pemphigus, folliculitis, skin inflammation caused by a cancer therapy optionally wherein the cancer therapy comprises an epidermal growth factor inhibitor, skin cancer, melanoma, squamous cell carcinoma, basal cell carcinoma, Merkel cell carcinoma, dermatofibrosarcoma protuberans, actinic keratosis, sebaceous carcinoma, a skin wound, a skin ulcer, a venous skin ulcer, a diabetic skin ulcer, arthritis, osteoarthritis, psoriatic arthritis, itching, pruritus, cholestatic pruritus, skin aging, skin wrinkles, a gram-positive bacterial skin infection, a staphylococcal skin infection, an S. aureus skin infection, acne, and advanced glycation end-product accumulation.

99. A method of preparing the pharmaceutical formulation of claim 1, comprising emulsifying and homogenizing a hydrophobic phase and an aqueous phase to produce said formulation.

100. The method of claim 98, wherein the skin condition is psoriasis.
